SEQUENCE LISTING (8 sequences)

SEQ ID NO: 1 - DNA, 1185 bp, Escherichia coli
  source 1..1185: mol_type = unassigned DNA
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgagttcccgaaagcgaactcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattga

SEQ ID NO: 2 - DNA, 1167 bp, Staphylococcus aureus
  source 1..1167: mol_type = unassigned DNA
atgacaataggtatcgataaaataaacttttacgttccaaagtactatgtagacatggctaaattagcagaagcacgccaagtagacccaaacaaatttttaattggaattggtcaaactgaaatggctgttagtcctgtaaaccaagacatcgtttcaatgggcgctaacgctgctaaggacattataacagacgaagacaaaaagaaaattggtatggtaattgtggcaactgaatcagcagttgatgctgctaaagcagccgctgttcaaattcacaacttattaggtattcaaccttttgcacgctgctttgaaatgaaagaagcttgttatgctgcaacaccagcaattcaattagctaaagattatttagcaactagaccgaatgaaaaagtattagttattgctacagatacagcacgttatggattgaactcaggcggcgagccaacacaaggtgctggcgcagttgcgatggttattgcacataatccaagcattttggcattaaatgaagatgctgttgcttacactgaagacgtttatgatttctggcgtccaactggacataaatatccattagttgatggtgcattatctaaagatgcttatatccgctcattccaacaaagctggaatgaatacgcaaaacgtcaaggtaagtcgctagctgacttcgcatctctatgcttccatgttccatttacaaaaatgggtaaaaaggcattagagtcaatcattgataacgctgatgaaacaactcaagagcgtttacgttcaggatatgaagatgctgtagattataaccgttatgtcggtaatatttatactggatcattatatttaagcctaatatcattacttgaaaatcgagatttacaagctggtgaaacaatcggtttattcagttatggctcaggttcagttggtgaattttatagtgcgacattagttgaaggctacaaagatcatttagatcaagctgcacataaagcattattaaataaccgtactgaagtatctgttgatgcatatgaaacattcttcaaacgttttgatgacgttgaatttgacgaagaacaagatgctgttcatgaagatcgtcatattttctacttatcaaatattgaaaataacgttcgcgaatatcacagaccagagtaa

SEQ ID NO: 3 - DNA, 1278 bp, Staphylococcus aureus
  source 1..1278: mol_type = unassigned DNA
atgcaaagtttagataagaattttcgacatttatctcgtaaagaaaagttacaacaattggttgataagcaatggttatcagaagaacaattcgacattttactgaatcatccattaatcgatgaagaagtagccaatagtttaattgaaaatgtcatcgcgcaaggtgcattacccgttggattattaccgaatatcattgtggacgataaggcatatgttgtacctatgatggtggaagagccttcagttgtcgctgcagctagttatggtgcaaagctagtgaatcagactggcggatttaaaacggtatcttctgaacgtattatgataggtcaaatcgtctttgatggcgttgacgatactgaaaaattatcagcagacattaaagctttagaaaagcaaattcataaaattgcggatgaggcatatccttctattaaagcgcgtggtggtggttaccaacgtatagcgattgatacatttcctgagcaacagttactatctttaaaagtatttgttgatacgaaagatgctatgggcgctaatatgcttaatacgattttagaggccataactgcatttttaaaaaatgaatttccgcaaagcgacattttaatgagtattttatccaatcatgcaacagcgtccgttgttaaagttcaaggcgaaattgatgttaaagatttagcaaggggcgagagaactggagaagaggttgccaaacgaatggaacgtgcttctgtattggcccaagtagatattcatcgtgcagcaacacataataaaggtgttatgaatggcatacatgctgttgttttagcaacaggaaatgatacgcgtggtgcagaagcaagtgcgcatgcatacgcgagtcgtgacggacagtatcgtggtattgctacatggcgttacgatcaagatcgtcaacgattgattggtacaattgaagtgcctatgacattggcaatcgttggcggtggtacaaaagtattaccaattgctaaagcttcattagagctactaaatgtagagtcagcacaagaattaggtcatgtagttgctgccgttggtttagcgcaaaactttgcagcatgtcgcgcgcttgtgtcagaaggtattcaacaaggtcatatgagtttacaatataaatcattagctatcgttgtaggggcaaaaggtgatgaaattgctaaagtagctgaagctttgaaaaaagaaccccgtgcaaatacacaagcagcggaacatattttacaagaaattagacaacaataa

SEQ ID NO: 4 - DNA, 1332 bp, Saccharomyces cerevisiae
  source 1..1332: mol_type = unassigned DNA
atgtctctgccattcctgacgtctgcgccaggtaaggtgatcatcttcggcgagcactctgcggtgtacaataagccggccgtcgccgcctctgtgtctgcgttacgcacctacctgctgatcagcgaatcttctgcaccggacacgatcgagctggactttccggacatcagcttcaaccacaagtggagcatcaacgacttcaacgcgatcacggaggaccaggtgaacagccaaaagctggccaaagcccagcaagcaaccgacggtctgtctcaggagctggtgtctctgctggacccgctgttagcgcagttaagcgagagcttccattaccacgccgcgttctgcttcctgtacatgttcgtttgcctgtgcccgcacgcaaagaacatcaagttcagcctgaagagcacgctgccgattggcgcaggcttaggctctagcgcatctatcagcgtgagcctggcgctggcgatggcctatctgggtggcctgattggcagcaacgacctggagaaactgagcgaaaacgacaagcacatcgtgaaccagtgggcctttatcggcgagaagtgcattcatggcaccccgagcggcattgacaacgcagttgccacgtatggcaacgccctgctgttcgagaaagacagccacaacggcacgatcaacacgaacaacttcaagttcctggacgacttcccggcgatcccgatgattctgacctacacccgtatcccacgcagcaccaaggatttagtcgcccgcgtgcgtgttttagtcaccgaaaagttcccggaggtgatgaagccgatcctggacgcgatgggcgagtgcgcgctgcagggtctggagatcatgaccaagctgagcaagtgcaagggcaccgacgatgaggcggtggaaaccaacaatgagctgtacgagcagctgctggagctgatccgtatcaatcacggcctgctggtttctatcggtgtgtctcacccgggcctggaactgatcaaaaacctgagcgacgacctgcgcattggctctacgaaattaacgggtgcaggtggcggtggctgctctttaacgctgctgcgccgtgacattacgcaggagcaaatcgacagcttcaagaagaagctgcaggacgacttcagctacgagacgttcgagacggacctgggcggcacgggctgttgcctgctgagcgccaaaaatctgaacaaggacctgaagatcaaaagcctggtgttccagctgttcgaaaacaagacgaccacgaagcagcagatcgacgacctgttactgccgggtaacaccaatctgccgtggacgtcttaa

SEQ ID NO: 5 - DNA, 1356 bp, Saccharomyces cerevisiae
  source 1..1356: mol_type = unassigned DNA
atgagcgaattacgtgcattcagcgcgccaggtaaggcactgctggccggtggctacctggtgttagacaccaagtacgaggcgttcgtcgtcggcttatctgcccgtatgcatgcagttgcccacccgtatggtagcctgcagggctctgacaagttcgaagtgcgtgtgaagagcaagcagttcaaggacggcgagtggctgtaccacattagcccaaagagcggcttcatcccggttagcattggtggcagcaagaacccatttatcgagaaggtcattgccaacgtcttcagctacttcaagccgaatatggacgattactgcaaccgcaacctgttcgtcatcgacattttcagcgacgacgcgtaccacagccaagaggactctgttacggagcatcgtggtaaccgccgcctgagcttccacagccatcgcattgaggaggtgccgaagacgggtctgggttctagcgccggtttagttaccgtcttaacgacggcgttagcgagcttcttcgtgagcgacctggagaacaacgtggacaagtaccgcgaagtgattcataacctggcgcaggtggcacattgtcaggcccaaggtaagattggctctggttttgatgtggcagcggccgcctatggctctatccgctatcgccgctttccgccggccctgatcagcaatctgccggacatcggctctgcgacgtatggtagcaaactggcgcatctggtggacgaagaagactggaacatcaccattaagtctaatcacctgccgagcggcttaacgttatggatgggcgatatcaagaacggcagcgaaacggttaagctggtgcagaaagtgaaaaactggtacgacagccacatgccggaaagcctgaagatttacacggagctggaccacgccaatagccgtttcatggatggtctgagcaagctggaccgcctgcacgaaacccacgacgactacagcgaccaaatcttcgagagcctggagcgcaatgactgcacctgccagaagtacccggagatcacggaggtccgcgatgccgtggcaacgattcgccgtagcttccgcaaaattacgaaggagagcggcgcggatatcgaaccaccggtccagacgtctctgctggacgactgtcaaaccttaaagggcgtgttaacgtgcctgattccgggcgcgggtggttacgacgccattgccgtcatcacgaaacaggacgtcgatctgcgcgcacaaacggccaacgacaaacgtttcagcaaagtccaatggctggatgttacgcaggccgactggggtgttcgcaaggagaaggacccggaaacgtatctggataagtga

SEQ ID NO: 6 - DNA, 549 bp, Escherichia coli
  source 1..549: mol_type = unassigned DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 7 - DNA, 1635 bp, Populus alba
  source 1..1635: mol_type = unassigned DNA
atggaagctcgtcgttctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttgtttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgagtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaa

SEQ ID NO: 8 - DNA, 1191 bp, Saccharomyces cerevisiae
  source 1..1191: mol_type = unassigned DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatngaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

(19) United States
(12) Patent Application Publication
(10) Pub. No.: US 2026/0117262 A1
(43) Pub. Date: Apr. 30, 2026

(54) GENETICALLY ENGINEERED MICROBE FOR THE PRODUCTION OF TERPENES AND METHOD OF PRODUCTION THEREOF

(71) Applicant: CleanJoule, Inc., Salt Lake City, UT (US)

(72) Inventors: Younghwan Kim, Salt Lake City, UT (US); Bohea Lee, Salt Lake City, UT (US); Robert Andrew Price, Salt Lake City, UT (US)

(73) Assignee: CleanJoule, Inc., Salt Lake City, UT (US)

(21) Appl. No.: 19/054,097

(22) Filed: Feb. 14, 2025

Related U.S. Application Data
(60) Provisional application No. 63/713,932, filed on Oct. 30, 2024.

(51) Int. Cl. C12P 5/00; C12N 9/04; C12N 9/10; C12N 9/12; C12N 9/88; C12N 9/90; C12N 15/52

(52) U.S. Cl. CPC C12P 5/007; C12N 9/0006; C12N 9/1025; C12N 9/1029; C12N 9/1229; C12N 9/88; C12N 9/90; C12N 15/52; C12Y 203/01009; C12Y 203/0301; C12Y 207/04002; C12Y 402/03027; C12Y 503/03002

(57) ABSTRACT

A genetically engineered microbe capable of producing isoprene from glycerol includes at least a native nucleic acid sequence encoding at least a native enzyme capable of catalyzing one or more steps of a conversion from at least a carbon source to acetyl coenzyme A (A-CoA), at least a first heterologous nucleic acid sequence encoding at least a first enzyme of a mevalonate (MVA) pathway, and at least a second heterologous nucleic acid sequence encoding at least a second enzyme capable of catalyzing at least a terpene-producing chemical reaction, wherein the at least a carbon source includes glycerol.

### CROSS-REFERENCE TO RELATED APPLICATIONS

This application claims the benefit of priority of U.S. Provisional Application Ser. No. 63/713,932, filed on Oct. 30, 2024, and entitled “GENETICALLY ENGINEERED MICROBE FOR PRODUCTION OF ISOPRENE FROM ETHANOL AND METHOD OF PRODUCTION THEREOF”, the entirety of which is incorporated herein by reference.

### REFERENCE TO SEQUENCE LISTING

This specification includes a sequence listing submitted herewith, which includes the file entitled 1656-007USU1_Sequence_Listing.xml having the following size: 17,297 bytes, which was created on Feb. 11, 2025, the contents of which are incorporated by reference herein.

### FIELD OF THE INVENTION

The present invention generally relates to the field of metabolic engineering. In particular, the present invention is directed towards a genetically engineered microbe for production of terpenes and a method of production thereof.

### BACKGROUND

Hydrocarbon materials such as without limitation terpenes are often defense-critical for a variety of applications including without limitation fuels, energetics, textiles, polymers, resins, etc. A particular example of terpenes is isoprene, which is an important commodity chemical used in a wide range of industrial applications, from the production of synthetic rubber for tires and coatings to uses in adhesives and development of specialty elastomers. Terpenes are currently traded at a scale of the millions of metric tons per year, with the majority of supply coming from outside of the continental United States (CONUS). The production of these terpenes often relies on organic synthesis and/or distillation and purification of petroleum products. These processes are often plagued by high costs (e.g., due to the use of precious-metal catalysts), low yields, competing side reactions, geographical restrictions, extensive land use, insecure supply chains, and/or lack of sustainability, among other drawbacks.

Biosynthesis of terpenes, while showing some progress, often has a limited yield due to a low carbon efficiency, which may be caused by inefficient metabolic pathways. The role of genetic and metabolic engineering towards improving the performance of such biosynthetic routes remains underexplored.

### SUMMARY OF THE DISCLOSURE

In an aspect, a genetically engineered microbe capable of producing a terpene from glycerol is described. The genetically engineered microbe includes at least a native nucleic acid sequence encoding at least a native enzyme. The at least a native enzyme is capable of catalyzing one or more steps of a conversion from at least a carbon source to acetyl coenzyme A (A-CoA). The genetically engineered microbe further includes at least a first heterologous nucleic acid sequence encoding at least a first enzyme of a mevalonate (MVA) pathway. The genetically engineered microbe further includes at least a second heterologous nucleic acid sequence encoding at least a second enzyme. The at least a second enzyme is capable of catalyzing at least a terpene-producing chemical reaction.

In one or more the genetically engineered microbe comprises a genetically engineered bacterium. In some cases, the genetically engineered bacterium may include Vibrio such as without limitation Vibrio natriegens.

In one or more embodiments, the at least a carbon source may include glycerol. In some cases, glycerol may have a concentration of at least 0.1 g/L and no greater than 10 g/L. In one or more embodiments, the at least a carbon source may further include glucose. In some cases, glucose may have a concentration of at least 0.1 g/L and no greater than 10 g/L. In one or more embodiments, the at least a carbon source may further include maltose. In some cases, glucose may have a concentration of at least 0.1 g/L and no greater than 10 g/L.

In one or more embodiments, the at least a first heterologous nucleic acid sequence may include one or more genes such as without limitation an atoB gene, an mvaS gene, an mvaA gene, an mk gene, a pmk gene, an idi gene, a pmd gene, and/or the like. In one or more embodiments, the at least a first heterologous nucleic acid sequence may include one or more genes such as without limitation an ERG10 gene, an ERG13 gene, an HMG1, an HMG2 gene, an ERG12 gene, an ERG8 gene, an MVD1 gene, an IDI1 gene, and/or the like.

In one or more embodiments, the at least a first enzyme includes at least a functional fragment of one or more enzymes, such as without limitation 3-hydroxy-3-methylglutaryl-coenzyme A reductase, acetyl-CoA acetyltransferase, 3-hydroxy-3-methylglutaryl-CoA synthase, HMG-CoA reductase, mevalonate kinase, phosphomevalonate kinase, diphosphomevalonate decarboxylase, and/or isopentenyl diphosphate isomerase, among others.

In one or more embodiments, the at least a second heterologous nucleic acid sequence may include an ispS gene. The ispS gene may accordingly encode at least a functional fragment of isoprene synthase (IspS).

In one or more embodiments, the terpene-producing chemical reaction may utilize dimethylallyl pyrophosphate (DMAPP) as a reactant.

In one or more embodiments, at least a metabolic pathway of the genetically engineered microbe, such as without limitation the MVA pathway, may be induced by isopropyl β-D-thiogalactopyranoside (IPTG) having a concentration of at least 0.05 millimolar and no greater than 0.3 millimolar.

In another aspect, a method of producing a terpene from glycerol is described. The method includes culturing a genetically engineered microbe under suitable conditions. The genetically engineered microbe includes at least a native nucleic acid sequence encoding at least a native enzyme. The at least a native enzyme is capable of catalyzing one or more steps of a conversion from at least a carbon source to acetyl coenzyme A (A-CoA). The genetically engineered microbe further includes at least a first heterologous nucleic acid sequence encoding at least a first enzyme of a mevalonate (MVA) pathway. The genetically engineered microbe includes at least a second heterologous nucleic acid sequence encoding at least a second enzyme. The at least a second enzyme is capable of catalyzing at least a terpene-producing chemical reaction. The method further includes providing a substrate to the cultured genetically engineered microbe.

In one or more the genetically engineered microbe comprises a genetically engineered bacterium. In some cases, the genetically engineered bacterium may include Vibrio such as without limitation Vibrio natriegens.

In one or more embodiments, the at least a carbon source may include glycerol. In some cases, glycerol may have a concentration of at least 0.1 g/L and no greater than 10 g/L. In one or more embodiments, the at least a carbon source may further include glucose. In some cases, glucose may have a concentration of at least 0.1 g/L and no greater than 10 g/L. In one or more embodiments, the at least a carbon source may further include maltose. In some cases, glucose may have a concentration of at least 0.1 g/L and no greater than 10 g/L.

In one or more embodiments, the at least a first heterologous nucleic acid sequence may include one or more genes such as without limitation an atoB gene, an mvaS gene, an mvaA gene, an mk gene, a pmk gene, an idi gene, a pmd gene, and/or the like. In one or more embodiments, the at least a first heterologous nucleic acid sequence may include one or more genes such as without limitation an ERG10 gene, an ERG13 gene, an HMG1, an HMG2 gene, an ERG12 gene, an ERG8 gene, an MVD1 gene, an IDI1 gene, and/or the like.

In one or more embodiments, the at least a first enzyme includes at least a functional fragment of one or more enzymes, such as without limitation 3-hydroxy-3-methylglutaryl-coenzyme A reductase, acetyl-CoA acetyltransferase, 3-hydroxy-3-methylglutaryl-CoA synthase, HMG-CoA reductase, mevalonate kinase, phosphomevalonate kinase, diphosphomevalonate decarboxylase, and/or isopentenyl diphosphate isomerase, among others.

In one or more embodiments, the at least a second heterologous nucleic acid sequence may include an ispS gene. The ispS gene may accordingly encode at least a functional fragment of isoprene synthase (IspS).

In one or more embodiments, the terpene-producing chemical reaction may utilize dimethylallyl pyrophosphate (DMAPP) as a reactant.

In one or more embodiments, at least a metabolic pathway of the genetically engineered microbe, such as without limitation the MVA pathway, may be induced by isopropyl β-D-thiogalactopyranoside (IPTG) having a concentration of at least 0.05 millimolar and no greater than 0.3 millimolar.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1A is an exemplary scheme of a metabolic pathway that converts glycerol to isoprene, including a plurality of enzymes catalyzing individual steps and representative genes encoding such enzymes;

FIG. 1B is an exemplary scheme of a metabolic pathway that converts glycerol to terpenes, including a plurality of enzymes catalyzing individual steps and representative genes encoding such enzymes;

FIGS. 2A-C are exemplary schemes containing an overview of metabolic pathways pertaining to biosynthesis of isoprene and chemical structures of each reactant, intermediate, and product related to their respective metabolic pathways;

FIGS. 3A-B are additional exemplary schemes of expanded metabolic pathways that are relevant for biosynthesis of isoprene; potential gene targets are also included, wherein the gene targets may be overexpressed or knocked down/knocked out to improve the yield of isoprene;

FIG. 4 is a flow diagram of an exemplary embodiment of a method for producing a terpene from glycerol using a genetically engineered microbe; and

FIGS. 5A-B are two exemplary embodiments of plasmid vector maps for pCJ1 and pCJ2 plasmids, respectively;

FIG. 6 is a set of exemplary experimental results with isoprene expression titers (quantified using peak areas, see left axis) measured as a function of isopropyl β-D-thiogalactopyranoside (IPTG) concentrations (0 mM, 0.01 mM, 0.1 mM, and 0.25 mM, respectively) and incubation time (20 hours and 44 hours, respectively); induction was conducted when the optical density at 600 nanometers (OD600, see right axis) of the sample reaches 0.85; and

FIG. 7 is a set of exemplary experimental results with isoprene expression titers (quantified using peak areas, see left axis) measured over two days, as a function of three carbon sources (i.e., glucose, glycerol, and maltose); associated OD600s of the samples are also shown (see right axis); microbes cultured under the same conditions without transfection by pCJ1 or pCJ2 plasmids were used as controls (marked as N/A); cell cultures were prepared separately using two types of growth media, i.e., LB3 and LBv2, for comparison.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to a genetically engineered microbe for production of terpenes and a method of production thereof. The genetically engineered microbe includes at least a native nucleic acid sequence encoding at least a native enzyme. The at least a native enzyme is capable of catalyzing one or more steps of a conversion from at least a carbon source to acetyl coenzyme A (A-CoA). The genetically engineered microbe further includes at least a first heterologous nucleic acid sequence encoding at least a first enzyme of a mevalonate (MVA) pathway. The genetically engineered microbe further includes at least a second heterologous nucleic acid sequence encoding at least a second enzyme. The at least a second enzyme is capable of catalyzing at least a terpene-producing chemical reaction.

Aspects of the present disclosure may be used to provide a sustainable means of producing isoprene, terpene(s), and similar value-added chemicals. Aspects of the present disclosure may be used to improve the carbon efficiency of biosynthetic techniques. Aspects of the present disclosure may be used to reduce the dependence on fossil fuels and mitigate the negative environmental impacts thereof. Exemplary embodiments illustrating aspects of the present disclosure are described below in the context of several specific examples.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (e.g., a step is performed by or with the assistance of a human).

For the purposes of this disclosure, including the claims, the phrase “at least some” means “one or more” and includes the case of only one. Thus, e.g., the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, e.g., the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, e.g., the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, e.g., the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, e.g., the phrase “multiple ABCs” means “two or more ABCs” and includes “two ABCs”. Similarly, e.g., the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, e.g., “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

For the purposes of this disclosure, an “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” may vary depending on the composition, the condition and its severity, the manner of administration, and the age of the subject to be treated.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of a neurological disorder). The term “therapeutic” includes the effect of mitigating deleterious clinical effects of certain processes (i.e., consequences of the process, rather than the symptoms of processes). As nonlimiting examples, a treatment may include (i) preventing a disease or condition from occurring in a subject, in particular, when such subject is predisposed to the condition but has not yet been diagnosed as having it; (ii) inhibiting a disease or condition, i.e., arresting its development; (iii) relieving a disease or condition, i.e., causing regression of the disease or condition; or (iv) relieving the symptoms resulting from a disease or condition, i.e., relieving pain without addressing the underlying disease or condition.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.”), and the like, is merely intended to better illustrate the invention and does not indicate a limitation on the scope of the invention unless specifically so claimed.

While the invention has been described in connection with what is presently considered to be the most practical and embodiments thereof are further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

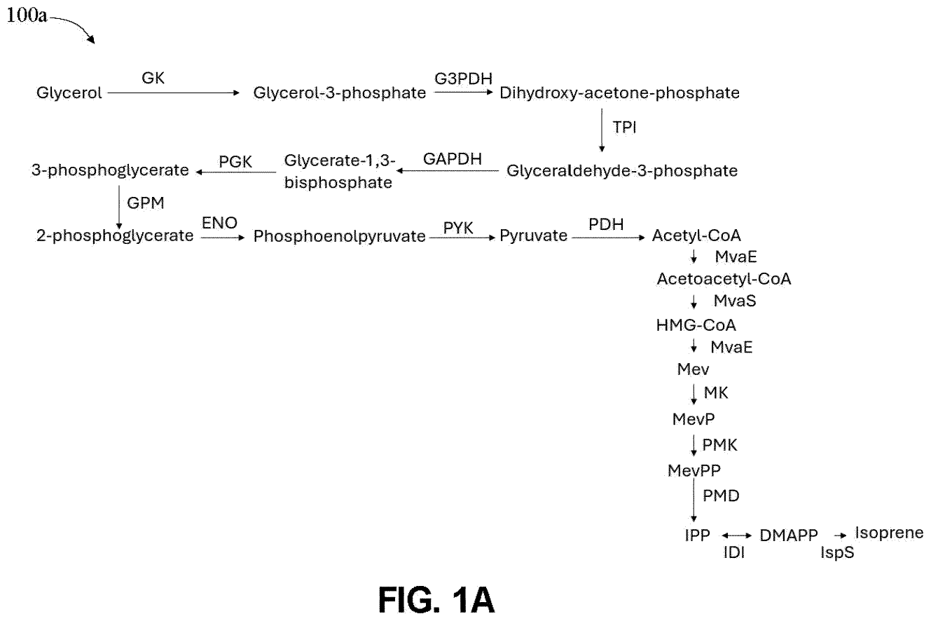

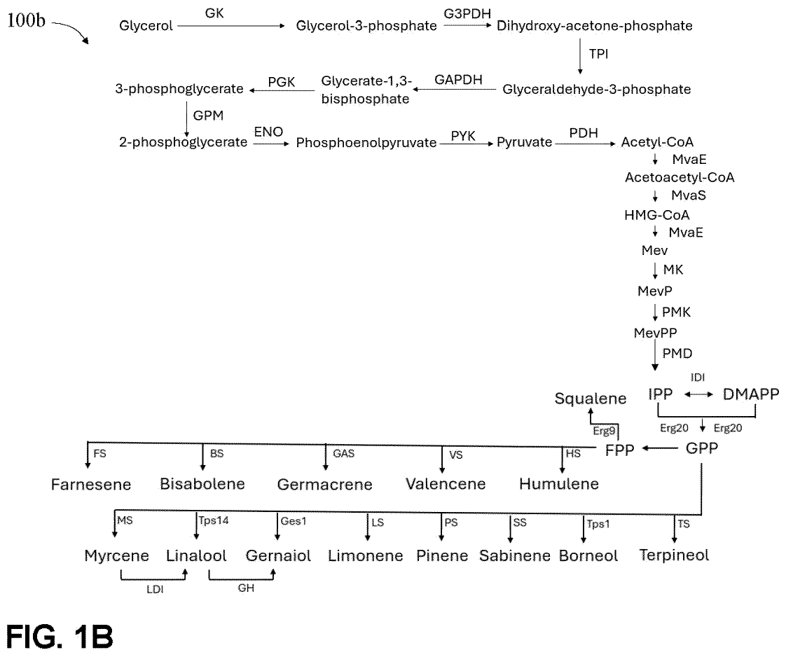

Referring now to FIG. 1A, a scheme 100a representing an exemplary metabolic pathway that converts glycerol to a terpene is illustrated. For the purposes of this disclosure, “isoprene”, also known as 2-methyl-1,3-butadiene, is a volatile organic compound with the chemical formula C5H8, a structural formula of CH2=CHC(CH3)=CH2, and a normal boiling point of 34° C. Isoprene may be considered a “hemiterpene” (i.e., half of a terpene), and the details of terpenes will be described below in this disclosure. Isoprene is a key building block for synthesizing natural rubber and various other polymers. Additionally, isoprene may be used in the manufacture of synthetic rubber, adhesives, and elastomers, as well as in the production of pharmaceuticals and other specialty chemicals. Isoprene may be produced naturally by plants and trees, particularly in large amounts by the rubber tree (i.e., Hevea brasiliensis). It may also be produced industrially through the thermal cracking of petroleum-derived naphtha or, in some cases, through biotechnological methods using genetically engineered microbes, as described in this disclosure. In some cases, isoprene may be synthesized using industrial organic chemistry, via synthetic routes such as an acetone/acetylene route, a propylene dimer route, an isoamylene route, an isopentane route, or an isobutylene/formaldehyde route, among others.

With continued reference to FIG. 1A, for the purposes of this disclosure, a “terpene” is an organic compound with the general molecular formula (C5H8)n, often characterized by a structure that contains repeating isoprene units. Terpenes are produced predominantly by plants, particularly conifers, and certain insects, and may be involved in various biological functions, including defense mechanisms and communication. Terpenes may be classified as monoterpenes (n=2), sesquiterpenes (n=3), diterpenes (n=4), and higher terpenes (n>4), based on the number of isoprene units they contain. Monoterpenes (C10H16) may include without limitation limonene found in citrus peels, myrcene in hops and lemongrass, pinene in pine resin, linalool in lavender, and geraniol in rose oil, among others. Sesquiterpenes (C15H24) may include without limitation farnesene in apple coatings, humulene in hops, bisabolol in chamomile, caryophyllene in black pepper, and nerolidol in ginger and tea tree oil. Diterpenes (C20H32) may include without limitation taxadiene from yew trees, gibberellins as plant hormones, phytol in chlorophyll, steviol in Stevia leaves, and retinol (Vitamin A1) derived from β-carotene. Higher terpenes may include without limitation squalene (C30H50) in shark liver oil, lanosterol (C30H50O) in wool grease, tetraterpenes or tetraterpenoids including carotenoids (C40H56) such as beta-carotene in carrots, and rubber (polyterpene) from latex. Terpenes may serve as a biochemical precursor for a wide array of natural products, including essential oils, resins, and various pharmacologically active compounds, making them valuable in industries such as pharmaceuticals, perfumery, and agriculture. Isoprene belongs to a broader class of molecules called isoprenoids, and similarly, terpenes belong to a broader class of molecules called terpenoids, though it is worth noting that these terminologies may often be used interchangeably. An isoprenoid may include a molecule that is derived from isoprene, and similarly, a “terpenoid” may include a molecule that is derived from a terpene, consistent with details described above. Isoprenoids and terpenoids include a large and diverse class of naturally occurring organic compounds. Isoprenoids/terpenoids may also be synthesized through either the MEP pathway or the MVA pathway, as described in detail above. Isoprenoids/terpenoids may play essential roles in various biological processes, including without limitation cellular membrane structure (as cholesterol), photosynthesis (as carotenoids), and growth regulation (as gibberellins). Isoprenoids/terpenoids may also be used in pharmaceuticals, fragrances, and biofuels due to their diverse chemical properties and biological activities.

With continued reference to FIG. 1A, it is worth noting that the invention described in this disclosure is not limited to production of isoprene only. As a nonlimiting example, the precursor for producing isoprene, such as without limitation isopentenyl diphosphate (IPP) and/or dimethylallyl diphosphate (DMAPP), may also be a building block for other isoprenoids, terpenes, or terpenoids; therefore, an accelerated biosynthesis of isoprene may contribute to an increase in yield for producing these isoprenoids, terpenes, or terpenoids. Specifically, in some cases, products of the MVA pathway such as DMAPP and IPP may be converted to geranyl diphosphate (GPP), which may be further converted to monoterpenes, etc. In some cases, products of the MVA pathway such as DMAPP and IPP may be converted to farnesyl diphosphate (FPP), which may be further converted to sesquiterpenes, triterpenes, and/or carotenoids, among others. A person of ordinary skill in the art, upon reviewing the entirety of this disclosure, will be able to recognize how the invention described herein may be extended to other related applications beyond isoprene synthesis.

With continued reference to FIG. 1A, scheme 100a is implemented within a genetically modified microbe. For the purposes of this disclosure, a “genetically engineered microbe” is a microbe whose genetic material has been manipulated to alter one or more of its hereditary traits. Such manipulation may include without limitation inserting, deleting, or otherwise modifying one or more specific DNA sequences; as a result of such manipulation, a genetically modified microbe may exhibit one or more different traits in its structure, function, or the like, compared to its naturally occurring counterparts. For the purposes of this disclosure, a “microbe” is a microscopic organism, including bacteria, archaea, fungi, protozoa, viruses, and/or the like, that is capable of being utilized in one or more aspects of a biotechnological application. A microbe may be characterized by its ability to perform certain biological processes, such as, without limitation, fermentation, gene expression, and metabolite production, among others. A microbe may be harnessed for purposes such as without limitation recombinant protein production, bioremediation, synthesis of pharmaceuticals, and development of biofuels, among others, due to their diverse metabolic capabilities and ease of genetic manipulation.

With continued reference to FIG. 1A, in one or more embodiments, the genetically engineered microbe may include a genetically engineered bacterium of fungus. In some cases, the genetically engineered microbe may include Escherichia including Escherichia coli (or E. coli), Vibrio including Vibrio natriegens (or V. natriegens), Saccharomyces including Saccharomyces cerevisiae (or S. cerevisiae), Aspergillus including Aspergillus niger, Aspergillus nidulans, and/or Aspergillus oryzae, Yarrowia including Yarrowia lipolytica (Y. lipolytica), Bacillus including Bacillus subtilis (B. subtilis), Clostridium including Clostridium ljungdahlii (C. ljungdahlii), and/or Pseudomonas including Pseudomonas putida (P. putida), among others.

With continued reference to FIG. 1A, for the purposes of this disclosure, Vibrio natriegens (V. natriegens) is a fast-growing, gram-negative marine bacterium. V. natriegens has recently gained prominence in biotechnology due to its exceptionally rapid doubling time of less than 10 minutes under suitable conditions. The fast growth, genetic tractability, high transformation efficiency, and ability to thrive in simple media of V. natriegens, among other traits, make it a suitable organism for various applications, including synthetic biology, recombinant protein production, metabolic engineering, and high-throughput screening, among others.

With continued reference to FIG. 1A, in some cases, creating a genetically engineered microbe may involve using recombinant DNA technology, CRISPR-Cas9, gene cloning, and/or other molecular biology techniques to achieve desired phenotypic changes. For the purposes of this disclosure, “recombinant DNA technology” is a type of technology that involves a manipulation of DNA sequences to create new genetic combinations not found in nature. Recombinant DNA technology may include techniques such as gene cloning, insertion of DNA fragments from one organism into another, and/or use of vectors such as plasmids to transfer a genetic material. Recombinant DNA technology may be used to express new traits or produce biological products such as proteins, enzymes, and hormones and may play a pivotal role in fields such as genetic engineering, biotechnology, and pharmaceutical development.

With continued reference to FIG. 1A, for the purposes of this disclosure, a “plasmid” is a circular, double-stranded DNA molecule distinct from a cell's chromosomal DNA and capable of autonomous replication. A plasmid may be used as a vector for insertion, expression, and propagation of foreign genes within a host organism. Such vectors may include specific sequences for an origin of replication, selectable markers, and cloning sites, enabling manipulation and study of genetic material for applications in research, biotechnology, and therapeutic development.

With continued reference to FIG. 1A, for the purposes of this disclosure, “CRISPR-Cas9” is a genetic engineering tool, derived from a bacterial immune defense mechanism, that includes Clustered Regularly Interspaced Short Palindromic Repeats (CRISPR) sequences and the CRISPR-associated protein 9 (Cas9) enzyme and allows for precise, targeted modification of DNA within an organism. CRISPR-Cas9 may be programmed with a guide RNA to target a specific DNA sequence, enabling Cas9 enzyme to create double-strand breaks at one or more precise locations, thereby facilitating insertion, deletion, or modification of genetic material.

With continued reference to FIG. 1A, the genetically engineered microbe includes at least a native nucleic acid sequence. For the purposes of this disclosure, a “native” nucleic acid sequence is a nucleic acid sequence that is naturally present within a cell. For the purposes of this disclosure, a “nucleic acid sequence” is a sequence of nucleotides that contains genetic information encoding a peptide. “Nucleic acid sequence” and “gene” may be used interchangeably throughout this disclosure. A nucleic acid sequence may include a coding sequence that encodes a peptide and one or more regulatory elements that regulate the expression of the coding sequence. Exemplary embodiments of a regulatory element may include a promoter, an enhancer, a silencer, an insulator, an operator, and a response element, among others. For the purposes of this disclosure, a “promoter” is a DNA sequence where an RNA polymerase binds to initiate transcription. For the purposes of this disclosure, an “enhancer” is a distant element of DNA sequence that increases transcription rates by interacting with a promoter via DNA looping. For the purposes of this disclosure, a “silencer” is a DNA sequence that represses transcription when bound by specific proteins. For the purposes of this disclosure, an “insulator” is a DNA sequence that prevents the interaction between one or more enhancers and one or more promoters of neighboring genes. For the purposes of this disclosure, an “operator” is a DNA segment that regulates the transcription of adjacent genes. For the purposes of this disclosure, a response element is a DNA sequence that responds to external signals, allowing genes to be turned on or off in response to environmental changes. These regulatory elements may work together to ensure precise gene expression necessary for proper cellular function.

With continued reference to FIG. 1A, in one or more embodiments, a nucleic acid sequence may include one or more operons. For the purposes of this disclosure, an “operon” is a functioning unit of DNA containing a cluster of genes under the control of a single promoter. It is commonly found in prokaryotes such as bacteria. These genes are transcribed together into a single messenger RNA strand and typically encode proteins that work together in a specific biological pathway. An operon may include a regulatory element, such as an operator as described above, where an activator or repressor protein may bind to increase or inhibit transcription. An operon may include one or more regulatory genes that encode one or more such activator or repressor proteins. A nonlimiting example of operon may include the lac operon, which regulates lactose metabolism in E. coli.

With continued reference to FIG. 1A, the at least a native gene encodes at least a native enzyme. For the purposes of this disclosure, a “native” enzyme is an enzyme that is synthesized by expressing one or more native genes within a cell. For the purposes of this disclosure, an “enzyme” is a biological catalyst, often a protein, having a three-dimensional structure specifically tailored for fitting a substrate or reactant and catalyzing a chemical reaction therefrom. Enzymes are often characterized by their high catalytic activity, high specificity toward substrates, and sensitivity to environmental factors such as temperature and pH. A substrate may interact with an enzyme (and its active/binding site) via a lock-and-key mechanism or an induced fit. For the purposes of this disclosure, a “catalyst” is a chemical capable of accelerating a chemical reaction by lowering at least an activation barrier along a reaction coordinate and increasing at least a rate constant associated with the at least an activation barrier. In some cases, to perform a catalytic function, a catalyst may first be consumed by one or more reactants to form one or more intermediates, then be regenerated as the one or more intermediates are converted to one or more products. In some cases, one or more reactants may bind to a catalyst, participate in a chemical reaction, then dissociate from the catalyst as one or more products. The catalytic function of a catalyst or enzyme may be described using mathematical tools such as Arrhenius equation, Eyring equation, Michaelis-Menten equation, Lineweaver-Burk equation, among others, as deemed suitable by a person of ordinary skill in the art upon reviewing the entirety of this disclosure.

With continued reference to FIG. 1A, the at least a native enzyme is capable of catalyzing one or more steps of a conversion from at least a carbon source to acetyl coenzyme A (A-CoA). For purposes of this disclosure, a “carbon source” is a molecule or collection of molecules, that can be broken down to provide carbon atoms for the biosynthesis of cellular components and/or energy production. For example, without limitation, at least a carbon source may include glucose, fatty acids, and/or amino acids. In an embodiment, at least a carbon source may be broken down through various metabolic pathways, such as for example, glycolysis and/or alcohol metabolism. “A-CoA,” for purposes of this disclosure, is a metabolic intermediate that functions as a donor of acetyl groups in various biochemical reactions. A-CoA may include a thioester formed from coenzyme A and an acetyl group (CH3CO), and may play a pivotal role in cellular metabolism, energy production, and biosynthetic pathways. A-CoA consists of an acetyl group (CH3CO—) covalently bonded to coenzyme A (CoA), which is a derivative of the B-vitamin pantothenic acid. The acetyl group is attached to the sulfur atom of the thiol (—SH) group of coenzyme A via a high-energy thioester bond, making A-CoA a useful molecule for a variety of biochemical processes.

With continued reference to FIG. 1A, in an embodiment, the at least a native enzyme may be specific to the particular substrate, of the at least a carbon source, involved in the conversion from at least a carbon source to A-CoA. Further, in an embodiment, the active site of the at least a native enzyme may require a high affinity for the substrate of the at least a carbon source. For example, and without limitation, in an embodiment wherein the substrate is ethanol the at least a native enzyme may include alcohol dehydrogenase (ADH). The ADH may catalyze the oxidation of ethanol to acetaldehyde, with the reduction of NAD+ to NADH. ADH's enzyme's active site may contain a zinc ion that plays a role in the oxidation of the alcohol group. Ethanol may bind to the active site, and NAD+ may facilitate the oxidation of the alcohol group to an aldehyde. From acetaldehyde (C2H4O), aldehyde dehydrogenase (ALDH) may act as the at least a native enzyme, wherein ALDH catalyzes the conversion of acetaldehyde to acetate (C2H3O2), a process that involves the reduction of NAD+ to NADH. The active site of ALDH may be highly specific for acetaldehyde. The enzyme may use NAD+ to oxidize the aldehyde group, which involves the nucleophilic attack by the NAD+ cofactor on the carbonyl carbon of the acetaldehyde. Further, from acetate, acetyl-CoA synthetase may act as the at least a native enzyme to catalyze the activation of acetate by forming A-CoA, a process that may require ATP and CoA. The active site of acetyl-CoA synthetase may have a binding site for acetate and CoA. In an embodiment, acetyl-CoA synthetase may facilitate the formation of a thioester bond between acetate and CoA, forming A-CoA.

With continued reference to FIG. 1A, in one or more embodiments, the at least a carbon source may include glycerol. In some cases, glycerol may have a concentration within a solution of at least 0.1 g/L and no greater than 10 g/L. In one or more embodiments, the at least a carbon source may further include glucose. In some cases, glucose may have a concentration within a solution of at least 0.1 g/L and no greater than 10 g/L. In one or more embodiments, the at least a carbon source may further include maltose. In some cases, maltose may have a concentration within a solution of at least 0.1 g/L and no greater than 10 g/L.

With continued reference to FIG. 1A, for purposes of this disclosure, “glycerol” is a three-carbon alcohol with a hydroxyl group (—OH) attached to each carbon, making it a triol. In an embodiment, glycerol may be found in its free form and/or as a part of larger molecules such as triglycerides. In an embodiment, glycerol may have a concentration within a solution of at least 0.1 g/L and no greater than 10 g/L, wherein a concentration within a solution of 0.1 g/L means that there is 0.1 grams of glycerol dissolved in each liter of solution and a concentration within a solution of 10 g/L means that there are 10 grams of glycerol dissolved in each liter of solution. In an embodiment, glycerol may have a concentration within a solution of 0.1 g/L. In an embodiment, glycerol may have a concentration within a solution of 1 g/L. In an embodiment, glycerol may have a concentration within a solution of 2 g/L. In an embodiment, glycerol may have a concentration within a solution of 5 g/L. in an embodiment, glycerol may have a concentration within a solution of 10 g/L.

With continued reference to FIG. 1A, for purposes of this disclosure, “glucose” is a six-carbon monosaccharide with an aldehyde group at one end, making it an aldose sugar. Glucose may be commonly found in the D-form in biological systems, and it can exist in a linear form and/or cyclic form. In an embodiment, glucose may have a concentration within a solution of at least 0.1 g/L and no greater than 10 g/L, wherein a concentration within a solution of 0.1 g/L means that there is 0.1 grams of glucose dissolved in each liter of solution and a concentration within a solution of 10 g/L means that there are 10 grams of glucose dissolved in each liter of solution. In an embodiment, glucose may have a concentration within a solution of 0.1 g/L. In an embodiment, glucose may have a concentration within a solution of 1 g/L. In an embodiment, glucose may have a concentration within a solution of 2 g/L. In an embodiment, glucose may have a concentration within a solution of 5 g/L. in an embodiment, glucose may have a concentration within a solution of 10 g/L.

With continued reference to FIG. 1A, for purposes of this disclosure, “maltose” is a disaccharide formed by two molecules of glucose. Maltose consists of two glucose units linked by an alpha-1,4-glycosidic bond. This bond connects the first carbon of one glucose molecule to the fourth carbon the second glucose molecule. In an embodiment, maltose may have a concentration within a solution of at least 0.1 g/L and no greater than 10 g/L, wherein a concentration within a solution of 0.1 g/L means that there are 0.1 grams of maltose dissolved in each liter of solution and a concentration within a solution of 10 g/L means that there are 10 grams of maltose dissolved in each liter of solution. In an embodiment, maltose may have a concentration within a solution of 0.1 g/L. In an embodiment, maltose may have a concentration within a solution of 1 g/L. In an embodiment, maltose may have a concentration within a solution of 2 g/L. In an embodiment, maltose may have a concentration within a solution of 5 g/L. in an embodiment, glycerol may have a concentration within a solution of 10 g/L.

With continued reference to FIG. 1A, the genetically engineered microbe further includes at least a first heterologous nucleic acid sequence encoding at least a first enzyme of a mevalonate (MVA) pathway. For purposes of this disclosure, a “heterologous” nucleic acid sequence is a foreign genetic sequence that has been introduced into the genetically engineered microbe. For purposes of this disclosure, a “MVA” pathway is a biochemical pathway that produces isoprenoids and sterols. In an embodiment, an MVA pathway may generate mevalonate, which is a 6-carbon molecule, from A-CoA, which may then be used to synthesize isoprenoid precursors. An MVA pathway may involve several enzymes, such as, without limitation, HMG-CoA reductase, mevalonate kinase, phosphomevalonate kinase, mevalonate diphosphate decarboxylase, and/or the like. The MVA pathway may eventually produce isopentenyl pyrophosphate (IPP), which is a building block for larger isoprenoid molecules.

With continued reference to FIG. 1A, in one or more embodiments, the at least a first heterologous nucleic acid sequence may include one or more genes such as without limitation an atoB gene, an mvaS gene, an mvaA gene, an mk gene, a pmk gene, an idi gene, a pmd gene, and/or the like. In one or more embodiments, the at least a first heterologous nucleic acid sequence may include one or more genes such as without limitation an ERG10 gene, an ERG13 gene, an HMG1, an HMG2 gene, an ERG12 gene, an ERG8 gene, an MVD1 gene, an IDI1 gene, and/or the like.

With continued reference to FIG. 1A, for purposes of this disclosure, an “atoB gene” is a gene that encodes the enzyme acetyl-CoA acetyltransferase. In an embodiment, acetyl-CoA acetyltransferase may play a role in the formation of acetoacetyl-CoA from two molecules of A-CoA. AtoB gene may kickstart the MVA pathway by contributing to the pool of metabolites that may be utilized in downstream steps.

With continued reference to FIG. 1A, for purposes of this disclosure, an “mvaS gene” is a gene that encodes mevalonate kinase (MvaK). MvaK catalyzes the conversion of mevalonate into mevalonate-5-phosphate, which is a key step in the MVA pathway. In an embodiment, mvaS gene may be crucial for further processing of mevalonate into other intermediates required for isoprenoid biosynthesis, such as IPP and DMAPP.

With continued reference to FIG. 1A, for purposes of this disclosure, an “mvaA gene” is a gene that encodes phosphomevalonate kinase. Phosphomevalonate kinase converts mevalonate-5-phosphate into mevalonate-5-diphosphate, which is a crucial intermediate in the MVA pathway. In an embodiment, mvaA may be involved in the phosphorylation steps that prepare the molecule for the production of isoprenoid precursors, further extending the biosynthesis process.

With continued reference to FIG. 1A, for purposes of this disclosure, an “mk gene” is a gene that encodes an enzyme involved in the phosphorylation of mevalonate. Specifically, mk gene may encode MvaK, which as previously discussed, catalyzes the conversion of mevalonate to mvalonate-5-phosphate. In some contexts, this gene may refer to a different kinase and/or isozyme that also plays a role in the activation of mevalonate within the MVA pathway.

With continued reference to FIG. 1A, for purposes of this disclosure, a “Pmk gene” is a gene that encodes phosphomevalonate kinase. Phosphomevalonate kinase is an enzyme that catalyzes the conversion of mevalonate-5-phosphate to mevalonate-5-diphosphate. Further, phosphomevalonate kinase may activate mevalonate into its dephosphorylated form, which is an essential step for the further biosynthesis of IPP and DMAPP in the MVA pathway.

With continued reference to FIG. 1A, for purposes of this disclosure, an “Idi gene” is a gene that encodes isopentenyl-diphosphate isomerase. Isopentenyl-diphosphate isomerase may convert IPP into DMAPP. Both IPP and DMAPP are isoprenoid precursors and may serve as building blocks for larger molecules in the isoprenoid biosynthetic pathway. In an embodiment, isopentenyl-diphosphate isomerase may be critical in ensuring that IPP can be converted into the correct form to continued downstream isoprenoid biosynthesis.

With continued reference to FIG. 1A, for purposes of this disclosure, a “pmd gene” is a gene that encodes phosphomevalonate dehydratase (pmd). Pmd may be responsible for the dehydration of mevalonate intermediates, which may contribute to the conversion of mevalonate-5-diphosphate into IPP and its derivatives. Pmd may serve a key role in producing the necessary molecules that serve as building blocks for terpenes, sterols, and/or other isoprenoid compounds.

With continued reference to FIG. 1A, for purposes of this disclosure, an “ERG10 gene” is a gene that encodes acetyl-CoA C-acetyltransferase. Acetyl-CoA C-acetyltransferase is an enzyme that catalyzes the first committed step in sterol biosynthesis. Specifically, acetyl-CoA C-acetyltransferase may catalyze the condensation of two molecules of A-CoA to form acetoacetyl-CoA. Acetoacetyl-CoA is the precursor for the production of HMG-CoA, which is a key intermediate in the MVA pathway. In an embodiment, overexpression of ERG10 can enhance the flux of carbon into the MVA pathway, leading to higher yields of isoprenoid products such as sterols and terpenes.

With continued reference to FIG. 1A, for purposes of this disclosure, an “ERG13 gene” is a gene that encodes hydroxymethylglutaryl-CoA synthase (HMG-CoA synthase). HMG-CoA synthase is an enzyme that catalyzes the condensation of A-CoA and acetoacetyl-CoA to form HMG-CoA (3-hydroxy-3-methylglutaryl-CoA). In an embodiment, HMG-CoA is a key intermediate in the MVA pathway, and its production is a rate-limiting step in the synthesis of mevalonate and subsequent isoprenoid biosynthesis. Further, in an embodiment, the overexpression of ERG13 may increase the concentration of HMG-CoA, thus boosting the production of downstream isoprenoid products.

With continued reference to FIG. 1A, for purposes of this disclosure, an “HMG1 gene” and an “HMG2 gene” are genes that encode 3-hydroxy-3-methylglutaryl-CoA reductases (HMG-CoA reductases). HMG-CoA reductase is an enzyme that catalyzes the reduction of HMG-CoA to mevalonate. In an embodiment, HMG-CoA reductase is considered the rate-limiting enzyme in the MVA pathway. HMG-CoA reductase may actively determine the flow of metabolites through the MVA pathway, which may be crucial for producing mevalonate and, ultimately, isoprenoids. Both HMG1 gene and HMG2 genes play an important role in the regulation of the MVA pathway. In an embodiment, overexpression and/or optimization of the HMG1 and HMG2 genes can increase mevalonate production, thereby enhancing the yield of isoprenoid compounds, such as terpenes, biofuels, and/or the like.

With continued reference to FIG. 1A, for purposes of this disclosure, an “ERG12 gene” is a gene that encodes MvaK. As discussed previously, MvaK is an enzyme involved in the phosphorylation of mevalonate. For example, MvaK may phosphorylate mevalonate to form mevalonate-5-phosphate, which is an intermediate that is further processed to produce isoprenoid precursors such as IPP and DMAPP. In an embodiment, by overexpressing ERG12 gene it may increase the phosphorylation of mevalonate, which helps to drive the overall production of isoprenoids and/or sterols.

With continued reference to FIG. 1A, for purposes of this disclosure, an “ERG8 gene” is a gene that encodes farnesyl diphosphate synthase (FPPS). FPPS is an enzyme responsible for the synthesis of farnesyl pyrophosphate (FPP) from DMAPP and IPP. FPP is a crucial intermediate in the biosynthesis of sterols and/or other isoprenoids. The ERG8-encoded enzyme may play a central role in terpene and sterol biosynthesis by catalyzing the condensation of isoprenoid units to form FPP, which is later converted into larger isoprenoid molecules. In an embodiment, engineering the expression of ERG8 in microorganisms may enhance terpene production, which is useful for biofuels, pharmaceuticals, and/or other high-value isoprenoid products.

With continued reference to FIG. 1A, for purposes of this disclosure, an “MVD1 gene” is a gene that encodes mevalonate diphosphate decarboxylase. Mevalonate diphosphate decarboxylase is an enzyme that catalyzes the decarboxylation of mevalonate-5-diphosphate (MVDP) to form IPP. In an embodiment, engineering the expression of MVD1 can increase the supply of IPP, which may be crucial in producing terpenes, steroids, and/or other isoprenoid-derived compounds.

With continued reference to FIG. 1A, for purposes of this disclosure, an “IDI gene” is a gene that encodes isopentenyl-diphosphate isomerase 1 (IDI1). IDI1 is an enzyme that catalyzes the isomerization of IPP into DMAPP. The conversion of IPP to DMAPP is essential for isoprenoid synthesis because both are required for the formation of larger isoprenoid molecules such as sterols and terpenes. This reaction may ensure the proper balance of these intermediates for the downstream production of biologically important compounds. In an embodiment, the overexpression of IDI1 in engineered microorganisms may ensure a higher supply of DMAPP, facilitating the production of isoprenoids, including compounds such as coenzyme Q, aromas, and/or biofuels.

With continued reference to FIG. 1A, in one or more embodiments, the at least a first enzyme includes at least a functional fragment of one or more enzymes, such as without limitation 3-hydroxy-3-methylglutaryl-coenzyme A reductase, acetyl-CoA acetyltransferase, 3-hydroxy-3-methylglutaryl-CoA synthase, HMG-CoA reductase, mevalonate kinase, phosphomevalonate kinase, diphosphomevalonate decarboxylase, and/or isopentenyl diphosphate isomerase, among others. For purposes of this disclosure, at least a “functional fragment” of one or more enzymes, refers to a portion or segment of a larger enzyme protein that retains its catalytic function. For example, even though at least a functional fragment may not encompass the entire structure of the enzyme, it may still contain the critical regions or domains necessary for the enzyme's catalytic function. In an embodiment, at least a functional fragment may be sufficiently intact to perform essential functions of the enzyme, which may include binding to substrates, catalyzing a specific reaction, and/or interacting with other molecules in the pathway. Further, in some embodiments, the at least a functional fragment of one or more enzymes may include a truncated molecule.

With continued reference to FIG. 1A, in an embodiment, at least a functional fragment of 3-hydroxy-3-methylglutaryl-coenzyme A reductase (HMG-CoA reductase) may include the catalytic domain that is responsible for reducing HMG-CoA to mevalonate. In an embodiment, the full-length enzyme may additionally include regulatory regions that control enzyme activity, but the catalytic fragment may still catalyze the reduction step. Further, in an embodiment, at least a functional fragment of acetyl-CoA acetyltransferase (atoB) may include the region responsible for the A-CoA binding site and the active site involved in condensation of A-CoA to acetoacetyl-CoA. For example, the N-terminal portion of the protein may be sufficient to perform the catalytic function while omitting the C-terminal domain, which may be involved in substrate recognition and/or enzyme regulation. In an embodiment, at least a functional fragment of 3-hydroxy-3methylglutaryl-CoA synthase (HMG-CoA synthase) may include the catalytic core responsible for the condensation of A-CoA and acetoacetyl-CoA to form HMG-CoA. Further, in some embodiments, at least a functional fragment of HMG-CoA reductase (HMGCR) may include the membrane-associated catalytic domain. Such a functional fragment may retain the ability to reduce HMG-CoA to mevalonate but may lack the membrane-anchor and/or regulatory domain that is involved in the enzyme's activation and feedback regulation. In some embodiments, at least a functional fragment of MvaK may include the N-terminal and/or C-terminal portion, which is responsible for ATP binding and substrate binding. This may allow the at least a functional fragment of MvaK to retain its ability to phosphorylate mevalonate at the 5-position to form mevalonate-5-phosphate, without the entirety of the enzyme, namely portions that may be involved in protein-protein interactions. In an embodiment, at least a functional fragment of phosphomevalonate kinase may include the active site that binds mevalonate-5-phosphate and ATP to catalyze the formation of mevalonate-5-diphosphate. In an embodiment, a truncated form of phosphomevalonate kinase that includes a kinase domain may still be able to phosphorylate the mevalonate derivative, even if it lacks other structural elements. In an embodiment, at least a functional fragment of diphosphomevalonate decarboxylase may include the part of the enzyme responsible for decarboxylating mevalonate-5-diphosphate to form IPP. This may include the C-terminal portion of diphosphomevalonate decarboxylase, which may work independently of the larger protein. Further, in an embodiment, at least a functional fragment of isopentenyl diphosphate isomerase (IDI) may include the portion of IDI responsible for isomerization of IPP and DMAPP. The active site for the isomerization process may be located within a compact domain, which may function independently from other parts of the enzyme involved in regulation and/or folding.

With continued reference to FIG. 1A, the genetically engineered microbe further includes at least a second heterologous nucleic acid sequence encoding at least a second enzyme. The at least a second enzyme is capable of catalyzing at least a terpene-producing chemical reaction. For purposes of this disclosure, a “terpene-producing chemical reaction” refers to a biochemical process that leads to the formation of terpenes. In an embodiment, the at least a second enzyme encoded by the second heterologous nucleic acid sequence may be involved with any step of the terpene biosynthesis pathway. For example, and without limitation, this may include enzymes responsible for initial precursors or terpenes, cyclization reactions, and/or modification steps. Further, the at least a second enzyme may specifically include geranyl pyrophosphate synthase (GPPS), farnesyl pyrophosphate synthase (FPPS), terpene synthases (TPS), squalene synthase (SQS), isoprene synthase, and/or the like.

With continued reference to FIG. 1A, in an embodiment, the at least a second enzyme encoded by the second heterologous nucleic acid sequence may depend directly on the target product. For example, the target product may include isoprene and/or alternatively, terpene. In an embodiment, if the target product is isoprene the at least a second enzyme may include isopentenyl-diphosphate isomerase (IDI1) and isoprene synthase, wherein IDI1 facilitates the isomerization of isopentenyl pyrophosphate (IPP) to dimethylallyl (DMAPP), and isoprene synthase subsequently catalyzes the conversions of DMAPP into isoprene and pyrophosphate. In an embodiment, if the target product is a terpene, the at least a second enzyme may include IDI1 and one or more prenyltransferases, wherein IDI1 facilitates the isomerization of isopentenyl pyrophosphate (IPP) to dimethylallyl pyrophosphate (DMAPP). A prenyltransferase, such as geranyl pyrophosphate synthase (GPPS), farnesyl pyrophosphate synthase (FPPS), or geranylgeranyl pyrophosphate synthase (GGPPS), may catalyze the sequential condensation of IPP and DMAPP to generate geranyl pyrophosphate (GPP), farnesyl pyrophosphate (FPP), or geranylgeranyl pyrophosphate (GGPP), respectively. These linear terpene precursors may subsequently undergo cyclization and modification by terpene synthases and tailoring enzymes, such as cytochrome P450 monooxygenases, methyltransferases, or glycosyltransferases, to produce a diverse range of monoterpenes, sesquiterpenes, diterpenes, and triterpenes.

With continued reference to FIG. 1A, in one or more embodiments, the at least a second heterologous nucleic acid sequence may include an ispS gene. The ispS gene may accordingly encode at least a functional fragment of isoprene synthase (IspS). For purposes of this disclosure, “ispS gene” is a gene that encodes isopentenyl diphosphate isomerase (often abbreviated as IDI or IPPI) and isoprene synthase. In an embodiment, the ispS gene may be involved in the MVA pathway and/or the non-mevalonate pathway, both of which are responsible for producing IPP and DMAPP. The isopentenyl diphosphate isomerase encoded by ispS may convert IPP to DMAPP by rearranging the structure of the molecule. Further, the conversion of IPP to DMAPP may help to regulate the balance of IPP and DMAPP, which may be critical for the production of various terpenes and terpenoids. In an embodiment, the at least a functional fragment of isoprene synthase may correspond to a part of the active site and/or a region of the enzyme essential for binding DMAPP and catalyzing the reaction to produce isoprene. For example, this may include C-terminal truncations of isoprene synthase. With continued reference to FIG. 1A, in one or more embodiments, the terpene-producing chemical reaction may utilize dimethylallyl pyrophosphate (DMAPP) as a reactant. For purposes of this disclosure, a “reactant” is a substance that undergoes a chemical reaction to produce one or more products. In an embodiment, the conversion of DMAPP into terpenes may be catalyzed by terpene synthases. For purposes of this disclosure, “terpene synthases” are a family of enzymes that catalyze the formation of cyclic structures or hydrocarbon backbones from linear precursors. Linear precursors may include, for example, GPP and/or FPP. In an embodiment, isoprene synthase may catalyze the direct conversion of DMAPP to isoprene. Other terpene synthases may catalyze reactions where DMAPP acts as part of a larger terpene backbone.

Now referring to FIG. 1B, illustrated is scheme 100b. In one or more embodiments, the MVA pathway may take various routes as a function of a target product. For example, a target product may include hemiterpenes, monoterpenes, triterpenes, and/or sesquiterpenes. Each target product may be synthesized at different points within the MVA pathway. For example, from IPP, phosphatase may catalyze a reaction generating isoprenol. In some cases, as discussed above IspS may convert DMAPP to isoprene.

With continued reference to FIG. 1B, in one or more embodiments, GPPS may catalyze the condensation of IPP and DMAPP to form GPP, a 10-carbon isoprenoid intermediate. From GPP the synthesis process may continue to synthesize various monoterpenes in the presence of various enzymes and/or be synthesized into FPP. In an embodiment, the various monoterpenes may include, but are not limited to myrcene, linalool, geraniol, limonene, pinene, sabinene, borneol, terpineol, and/or the like. From FPP triterpenes and/or sesquiterpenes may be synthesized in the presence of specific enzymes. In an embodiment, triterpenes may include squalene. Further, in some embodiments, sesquiterpenes may include farnesene, bisabolene, germacrene, valencene, and/or humulene. In an embodiment, the selective production of one or more monoterpenes, triterpenes, and/or sesquiterpenes may be catalyzed by at least a functional fragment of one or more enzymes. The one or more enzymes may include, without limitation, isoprene synthase (IspS), phosphatase (PP), myrcene synthase (MS), linalool synthase (Tsp14), geraniol synthase (GeS1), limonene synthase (LiS), pinene synthase (PS), sabinene synthase (SS), trehalose-6-P synthase (Tps1), terpineol synthase (TS), farnesene synthase (FS), bisabolene synthase (BS), germacrene synthase (GAS), valencene synthase (VS), humulene synthase (HS), farnesyl-diphosphate farnesyl transferase (Erg9), and/or myrcene isomerase (LD1), among others.

With continued reference to FIG. 1A, in one or more embodiments, at least a metabolic pathway of the genetically engineered microbe, such as without limitation the MVA pathway, may be induced by isopropyl β-D-thiogalactopyranoside (IPTG) having a concentration of at least 0.05 millimolar and no greater than 0.3 millimolar. In one or more embodiments, at least a metabolic pathway of the genetically engineered microbe, such as without limitation the MVA pathway, may be induced by isopropyl β-D-thiogalactopyranoside (IPTG) having a concentration of at least 0.01 millimolar and no greater than 0.5 millimolar. For purposes of this disclosure, a “metabolic pathway” refers to a series of enzymatic reactions that lead to the conversion of precursor molecules into specific products. For purposes of this disclosure, “IPTG” is a synthetic molecule used to induce expression of genes that are under the control of the lac operator. A “lac operator,” as used herein, is a regulatory sequence that controls gene expression. In an embodiment, IPTG may act by binding to the lac repressor, which normally blocks the transcription of genes, thereby displacing the repressor and allowing transcriptional activation of the gene. When used to induce gene expression, IPTG may effectively trigger the activation of genetically engineered pathways, enabling the production of enzymes required for the biosynthesis of key metabolites. In an embodiment, IPTG is a potent inducer that may be used to activate recombinant genes that have been inserted into microbial systems.

With continued reference to FIG. 1A, in an embodiment, the concentration of IPTG is relevant for tuning the level of gene expression in engineered microbes. The precise concentration of IPTG may affect the amount of protein expressed and, consequently, the efficiency of the metabolic pathway being induced. At lower concentrations, IPTG may induce moderate expression, which may be beneficial when higher metabolic flux is inhibitory and/or wasteful. Alternatively, at higher concentrations, more protein is produced, leading to a higher degree of metabolic pathway activation, which may increase the yield of the target product, such as terpenes and/or other isoprenoids. In an embodiment, control of gene expression is crucial because an overly high induction may lead to metabolic overload, improper folding of proteins, and/or the diversion of cellular resources away from growth, while too-low induction may not produce enough of the necessary enzymes to significantly increase pathway flux. In an embodiment, IPTG may have a concentration of 0.05 millimolar. In an embodiment, IPTG may have a concentration of 0.1 millimolar. In an embodiment, IPTG may have a concentration of 0.2 millimolar. In an embodiment, IPTG may have a concentration of 0.3 millimolar.

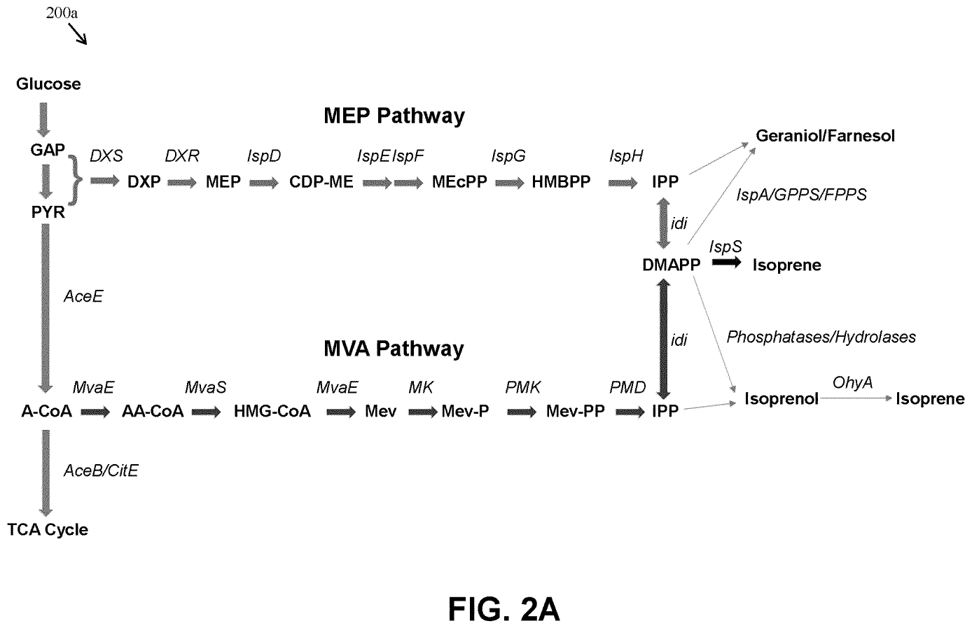

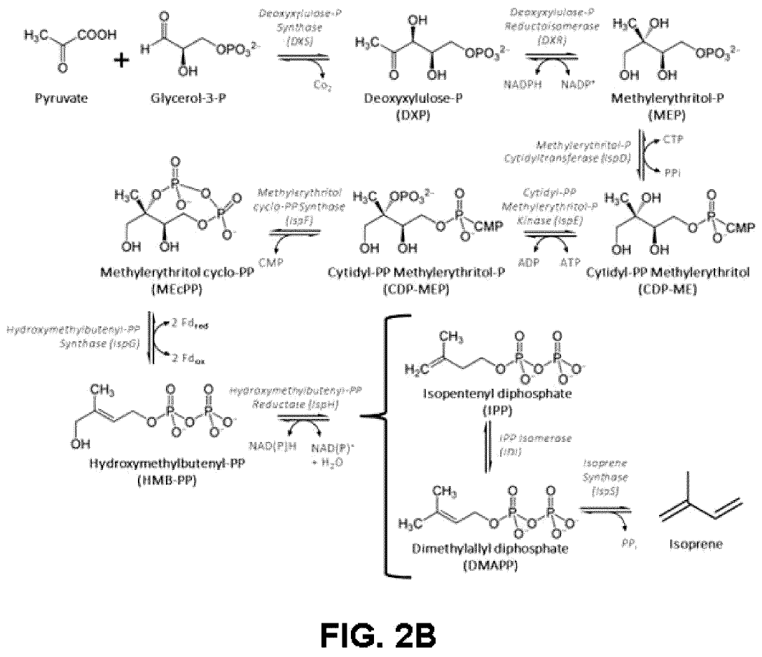

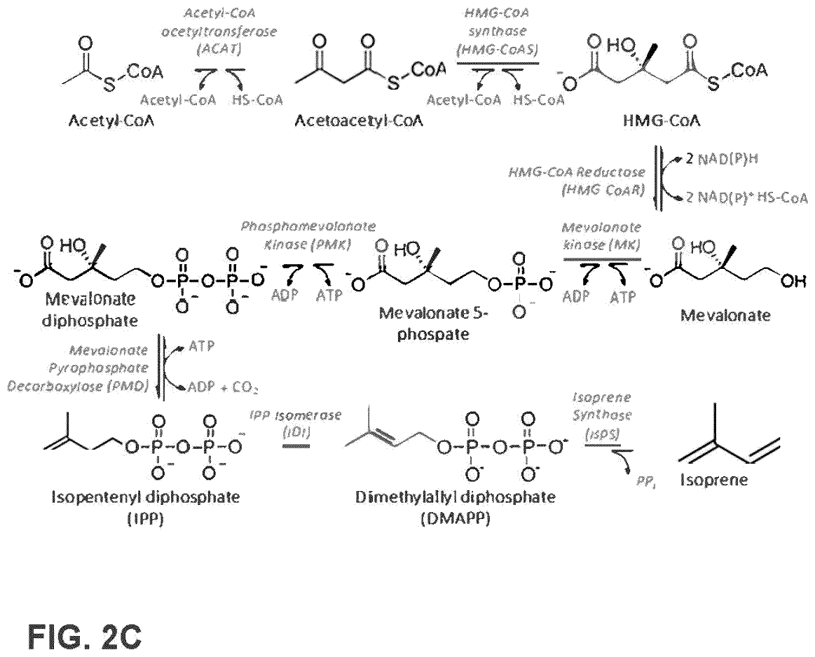

Referring now to FIG. 2A, scheme 200a illustrates several metabolic pathways pertaining to biosynthesis of isoprene, highlighting the substrate, product, and/or enzyme (catalyst) associated with each step. Scheme 200a includes part of the central metabolic pathway, i.e., the cellular respiration pathway, wherein glucose is broken down to feed the tricarboxylic acid (TCA) cycle. Conversion of glucose involves several key steps. Initially, glucose (C6H12O6) undergoes glycolysis by converting to two molecules of glyceraldehyde 3-phosphate (GAP). Such conversion is catalyzed by enzymes including hexokinase, phosphofructokinase, and aldolase. GAP is then further processed to form pyruvate (C3H3O3-), producing a net gain of 2 ATP and 1 NADH. Such conversion is catalyzed by enzymes such as glyceraldehyde 3-phosphate dehydrogenase and pyruvate kinase. Pyruvate is further converted to acetyl coenzyme A (A-CoA), releasing NADH and CO2. Such conversion is catalyzed by pyruvate dehydrogenase (PDH), which may include pyruvate dehydrogenase E1 component (encoded by gene AceE). A-CoA then enters the TCA cycle (not shown) and participates in reactions catalyzed by enzymes such as, without limitation, malate synthase (AceB) or ATP citrate lyase. It is worth noting that glucose is not the only viable carbon source for the invention described in this disclosure; alternatives such as sucrose, biomass, glycerol, ethanol, plant oils, or the like, may be used instead upon strategic engineering of metabolic pathways.

With continued reference to FIG. 2A, scheme 200a includes methylerythritol phosphate (MEP) and mevalonate (MVA) pathways for isoprene production. Both the MEP and the MVA pathways are connected to the central metabolic pathway and may lead to production of isoprene, consistent with details described above. For the purposes of this disclosure, the methylerythritol phosphate (MEP) pathway, also known as the non-mevalonate pathway, is a metabolic route for isoprenoid biosynthesis in bacteria, algae, and/or plant plastids. It begins with the formation of 1-deoxy-D-xylulose-5-phosphate (DXP) from pyruvate and glyceraldehyde-3-phosphate (GAP), which is catalyzed by 1-deoxy-D-xylulose-5-phosphate synthase (DXS). This step is one of the rate-determining steps of the MEP pathway. DXP is then converted to 2-C-methyl-D-erythritol-4-phosphate (MEP), which is catalyzed by 1-deoxy-D-xylulose 5-phosphate reductase (DXR). MEP is then converted to 4-diphosphocytidyl-2-C-methyl-D-erythritol (CDP-ME), which is catalyzed by 4-diphosphocytidyl-2-C-methyl-D-erythritol synthase (IspD). CDP-ME is further converted via two steps to 2-C-methyl-D-erythritol-2,4-cyclodiphosphate (MEcPP), which is catalyzed by 4-diphosphocytidyl-2-C-methyl-Derythritol kinase (IspE) and 2-C-methyl-D-erythritol 2,4-cyclodiphosphate synthase (IspF). MEcPP is then reduced to 4-hydroxy-3-methylbut-2-enyl diphosphate (HMBPP), which is catalyzed by (E)-4-Hydroxy-3-methylbut-2-enyl pyrophosphate synthase (IspG). HMBPP is then converted to isopentenyl diphosphate (IPP) and dimethylallyl diphosphate (DMAPP), which is catalyzed by (E)-4-Hydroxy-3-methylbut-2-enyl pyrophosphate reductase (IspH or HMBPP reductase). IPP and DMAPP may establish a chemical equilibrium and interconvert with one another, which is catalyzed by isopentenyl diphosphate isomerase (idi). DMAPP may be converted to isoprene, which is catalyzed by isoprene synthase (IspS). IspS may be derived from plants, as many plants such as kudzu (the Asian vine, Pueraria montana), aspen (Populus tremuloides), and poplar (Populus), among others, may naturally produce isoprene by the catalytic elimination of pyrophosphate from DMAPP. It is worth noting that, in order to utilize ethanol as a carbon source and the MVA pathway, the MEP pathway may sometimes be suppressed, at least to some extent, in order to direct the flux towards the MVA pathway. Chemical structures of each reactant, intermediate, and product of the MEP pathway are illustrated in FIG. 2B.

Still referring to FIG. 2A, for the purposes of this disclosure, the mevalonate (MVA) pathway is another metabolic route for isoprenoid biosynthesis, found in archaea, fungi, animals, and some bacteria. The MVA pathway starts with the conversion of A-CoA to acetoacetyl-coenzyme A (AA-CoA), which is catalyzed by acetoacetyl-coenzyme A thiolase or 3-hydroxy-3-methylglutaryl-coenzyme A reductase (MvaE, which may be encoded by gene ERG10). AA-CoA is then converted to 3-hydroxy-3-methylglutaryl-CoA (HMG-CoA), which is catalyzed by HMG-CoA synthase (MvaS, which may be encoded by gene ERG13). HMG-CoA is then reduced to mevalonate (Mev). This step is catalyzed by MvaE, which may be encoded by gene HMG1, HMG2, or HMG-CoA reductase (HMGR). This step is the rate-determining step of the MVA pathway. Mev is phosphorylated to mevalonate-5-phosphate (Mev-P), which is catalyzed by mevalonate kinase (MK), which could be encoded by ERG12. Mev-P is phosphorylated to form mevalonate-5-diphosphate (Mev-PP), which is catalyzed by phosphomevalonate kinase (PMK), which could be encoded by ERG8. Mev-PP is subsequently decarboxylated to produce IPP, which is catalyzed by mevalonate-5-diphosphate decarboxylase or diphosphomevalonate decarboxylase (PMD), which may be encoded by MVD1. IPP and DMAPP may establish a chemical equilibrium and interconvert with one another, which is catalyzed by idi, consistent with details described above. The idi enzyme may be encoded by IDI1. DMAPP may be converted to isoprene, which is catalyzed by IspS, consistent with details described above. In some cases, the MVA pathway may be further categorized into the “upper pathway”, which converts A-CoA to MVA, and the “lower pathway”, which produces DMAPP from MVA. In some cases, the MVA pathway may be introduced (i.e., cloned) into bacteria such as E. coli (which only has a native MEP pathway) as two synthetic operons, using a combination of bacterial and yeast enzymes. As a nonlimiting example, the lower pathway operon may be integrated into the chromosome while the upper pathway operon may be expressed on one or more plasmids, optionally alongside an IspS-encoding gene or the like. Chemical structures of each reactant, intermediate, and product of the MVP pathway are illustrated in FIG. 2C.

With continued reference to FIG. 2, scheme 200a includes a plurality of competing pathways that may limit the yield of isoprene production. IPP and/or DMAPP may be converted to geraniol and/or farnesol, which is catalyzed by enzymes such as without limitation geranyl diphosphate synthase (IspA), geranyl diphosphate synthase (GPPS), and/or farnesyl diphosphate synthase (FPPS). IPP and/or DMAPP may be converted to isoprenol, which is catalyzed by enzymes such as phosphatases and/or hydrolases including the Nudix hydrolase encoded by the NudB gene. As part of the central metabolic pathway, A-CoA may be converted to citrate to enter the tricarboxylic acid cycle, which is catalyzed by enzymes such as ATP citrate lyase including AclY, AclB, and/or AclA. These pathways compete with the formation of isoprene. In some cases, to improve the yield of isoprene, byproduct isoprenol may be converted back to isoprene, which may be catalyzed by enzymes such as 3-methyl-3-buten-1-ol dehydroxylase (OhyA) or the like.

Now referring to FIG. 2B, chemical structures of reactants, intermediates, and products of the MEP pathway are illustrated.

Now referring to FIG. 2C, chemical structures of reactants, intermediates, and products of the MVP pathway are illustrated.

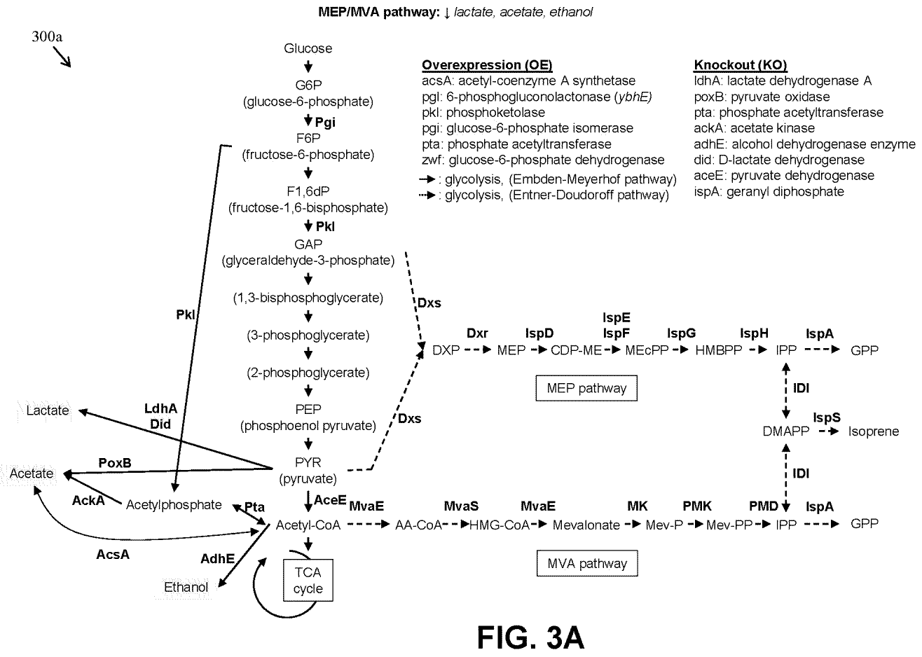

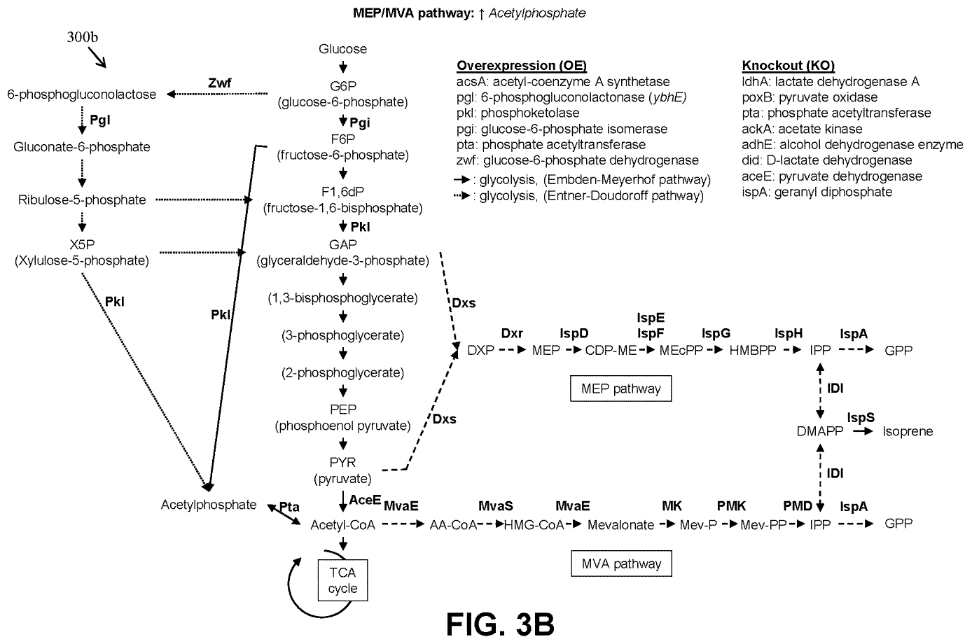

Referring now to FIGS. 3A-B, exemplary embodiments 300a-b of expanded metabolic pathways pertaining to isoprene biosynthesis are illustrated. FIGS. 3A-B include a plurality of potential gene targets. These gene targets and the activity thereof may be upregulated (for example, overexpressed), downregulated (for example, knocked down (KD)/knocked out (KO)), or otherwise modified, either individually or in combination, to increase the yield of isoprene.

With continued reference to FIGS. 3A-B, in one or more embodiments, the inherent activity of an enzyme may be improved by modifying the nucleic acid sequence that encodes the enzyme. Various techniques may be used to improve the inherent activity of an enzyme. As a nonlimiting example, directed evolution may be used to mimic natural selection in the lab by creating a large library of enzyme variants and selecting those with improved traits. As another nonlimiting example, site-directed mutagenesis may be used to introduce one or more specific mutations to enhance enzyme activity or stability. As another nonlimiting example, gene shuffling may be used to recombine segments of related genes to create new variants with enhanced properties. As another nonlimiting example, fusion proteins may be created by combining enzymes with other proteins to improve their function and/or stability. As a nonlimiting example, an enzyme such as OhyA or the like may be engineered to increase its inherent activity, thereby facilitating accumulation of isoprene while lifting the potentially toxic impact of isoprenol. As another nonlimiting example, an enzyme such MvaE or HMGR may be truncated from its naturally existing counterpart to improve its catalytic activity. As another nonlimiting example, one or more cofactors for one or more enzymes may be switched in order to improve the catalytic activity of an enzyme or enable fermentation conditions to be utilized that are more cost effective (e.g., with a lower oxygen requirement).

With continued reference to FIGS. 3A-B, in one or more embodiments, genes encoding enzymes such as 1-deoxy-D-xylulose-5-phosphate synthase (DXS), isoprene synthase (IspS), 3-hydroxy-3-methylglutaryl-coenzyme A reductase (HMG-CoA reductase or HMGR), 3-methyl-3-buten-1-ol dehydroxylase (OhyA) or OhyA-like enzymes, (E)-4-hydroxy-3-methylbut-2-enyl diphosphate synthase (IspG), 1-deoxy-D-xylulose 5-phosphate reductase (DXR), (E)-4-hydroxy-3-methylbut-2-enyl diphosphate reductase (IspH or HMBPP reductase), acetyl-CoA C-acetyltransferase/acetoacetyl-coenzyme A thiolase (MvaE, encoded by gene ERG10), HMG-CoA Synthase (MvaS, encoded by gene ERG13), acylating acetaldehyde dehydrogenase (A-ALD), among others, may be modified to increase the inherent activity of the enzyme, consistent with details described in the rest of this disclosure.

With continued reference to FIGS. 3A-B, in one or more embodiments, the activity of an enzyme may be modulated by increasing or upregulating an expression of a nucleic acid construct that encodes the enzyme. In some cases, a coding sequence may be configured to upregulate one or more genes encoding enzymes such as phosphatase, hydrolase, 3-methyl-3-buten-1-ol dehydroxylase (OhyA), transcription factor (encoded by UPC2), acetyl-CoA C-acetyltransferase/acetoacetyl-coenzyme A thiolase (MvaE, encoded by gene ERG10), mevalonate kinase (MK, encoded by ERG12), HMG-CoA synthase (MvaS, encoded by gene ERG13), pantothenate kinase (encoded by CAB1), dihydrolipoamide acetyltransferase component of the pyruvate dehydrogenase complex (encoded by LAT1), glucose-6-phosphate dehydrogenase (encoded by zwf), 6-phosphogluconolactonase (ybhE, encoded by pgl), phosphoketolase (encoded by pkl), glucose-6-phosphate isomerase (encoded by pgi), and phosphate acetyltransferase (encoded by pta), among others, as these genes encode enzymes that may contribute to the production and/or accumulation of isoprene. In some cases, regulation of a single gene may result in a regulation of multiple enzymes. As a nonlimiting example, when UPC2 is upregulated, ERG12 and ERG13 may also be overexpressed.

With continued reference to FIG. 3A-B, in one or more embodiments, in order to utilize ethanol as a carbon source for production of isoprene, the activity of an enzyme may be modulated by decreasing, attenuating, downregulating, or deleting the expression of a nucleic acid construct that encodes the enzyme. In some cases, a gene encoding an enzyme may be knocked down or knocked out. As nonlimiting examples, one or more genes encoding enzymes such as geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), pyruvate dehydrogenase (encoded by PDH) including pyruvate dehydrogenase E1 component (encoded by AceE), pyruvate oxidase (encoded by poxB), pyruvate decarboxylase (encoded by PDC), malate synthase (encoded by AceB or MLS1), citrate synthase (encoded by CIT1), ATP-citrate lyase including AclY, AclB, and AclA, glycerol-producing enzymes including cGPDH [40] and G3PP [41], ethanol-producing enzymes including invertase and zymase, alcohol dehydrogenase (encoded by ADH1), alcohol dehydrogenase enzyme (encoded by adhE), lactate-producing enzymes including lactate dehydrogenase such as lactate dehydrogenase A (encoded by IdhA) and D-lactate dehydrogenase (encoded by did), acetate-producing enzymes including acetyl coenzyme A (acetyl-CoA) hydrolases, acetate kinase (encoded by ackA), phosphatase, and hydrolase (for example, a hydrolase encoded by the NudB gene), among others, may be downregulated. However, it is worth noting that, in some cases, an enzyme may perform an essential function of a chassis and should not be completely knocked out or suppressed. As a nonlimiting example, farnesyl pyrophosphate synthetase encoded by ERG20 is essential to S. cerevisiae, and accordingly, specific sites within ERG20 may be mutated in order to downregulate (not eliminate) its function. As another nonlimiting example, a diploid strain may be used to knockout one copy of ERG20 gene in order to downregulate the activity of the enzyme it encodes. In some cases, an enzyme may not be essential to a chassis and may be knocked out without causing adverse effects. As a nonlimiting example, genes such as MLS1 and CIT1 may be knocked out in such manner.

With continued reference to FIG. 3A-B, in some cases, multiple upregulations and downregulations may be implemented simultaneously to achieve a synergic effect. As a nonlimiting example, gene targets such as zwf, pgl, and/or pkl may be upregulated simultaneously to utilize the Entner-Doudoroff pathway in addition to the Embden-Meyerhof pathway regulated by pgi and pkl, thereby increasing the carbon flux of biosynthesis and increasing the yield of acetylphosphate (see FIG. 3A, embodiment 300a). As another nonlimiting example, gene targets such as ackA-pta, poxB, and/or ldhA may be knocked down or knocked out simultaneously to suppress competing reactions that form acetate, lactate, and/or ethanol (see FIG. 3B, embodiment 300b). As another nonlimiting example, simultaneous upregulation of ALD5 and downregulation of ADH1 may be combined to prevent acetyl-CoA from converting to ethanol via fermentation. Table 1 summarizes exemplary gene targets for upregulation and downregulation for S. cerevisiae.

TABLE 1
Gene Targets for Improving Isoprene Production in S. cerevisiae.

| | | Type of Genetic | |
|---|---|---|---|
| | | Modification | |
| | | Note: | |
| | | Knockout (KO) | |
| | | Knockdown (KD) | |
| Organism | Gene | Overexpress (OE) | Rationale |
| S. cerevisiae | ERG20: farnesyl | KD or mutate | Essential Gene; |
| | pyrophosphate synthetase | specific sites | increase DMAPP |
| S. cerevisiae | BTS1: geranylgeranyl | KO/KD | Increase DMAPP |
| | diphosphate synthase | | |
| S. cerevisiae | UPC2: transcription | OE | Increase products of |
| | factor | | MVA pathway |
| S. cerevisiae | ERG10: acetyl-CoA C- | OE | Increase products of |
| | acetyltransferase | | MVA pathway |
| S. cerevisiae | ERG12: Mevalonate | OE | Increase products of |
| | kinase | | MVA pathway |
| S. cerevisiae | ERG13: HMG-CoA | OE | Increase products of |
| | synthase | | MVA pathway |
| S. cerevisiae | ALD5: aldehyde | OE | Increase flux from |
| | dehydrogenases | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | ADH1: alcohol | KO/KD | Increase flux from |
| | dehydrogenase | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | ACS1 and ACS2: acetyl- | OE | Increase flux from |
| | CoA synthetase | | acetaldehyde to |
| | | | acetyl-CoA instead |
| | | | of ethanol |
| | | | production. Could |
| | | | also work with fed |
| | | | ethanol to push |
| | | | towards acety-CoA |
| S. cerevisiae | MLS1: Malate synthase | KO/KD | Increase acetyl CoA |
| | | | pool |
| S. cerevisiae | CIT1: Citrate synthase | KO/KD | Increase acetyl CoA |
| | | | pool |
| S. cerevisiae | CAB1: Pantothenate | OE | Increase acetyl CoA |
| | kinase | | pool |
| S. cerevisiae | LAT1: dihydrolipoamide | OF | Increase acetyl CoA |
| | acetyltransferase | | pool |
| | component of the | | |
| | pyruvate dehydrogenase | | |
| | complex | | |
| E. coli | zwf: glucose-6-phosphate | OE | Increase carbon flux |
| | dehydrogenase | | to acetylphosphate |
| | | | (overexpression of |
| | | | the Entner- |
| | | | Doudoroff pathway) |
| E. coli | acsA: acetyl-coenzyme A | OE | Increase carbon flux |
| | synthetase | | from acetate to |
| | | | acetyl-CoA |
| E. coli | pgl: 6- | OE | Increase carbon flux |
| | phosphogluconolactonase | | to acetylphosphate |
| | (ybhE) | | (overexpression of |
| | | | the Entner- |
| | | | Doudoroff pathway) |
| E. coli | pkl: phosphoketolase | OE | Increase carbon flux |
| | | | to acetylphosphate |
| | | | (overexpression of |
| | | | the Entner- |
| | | | Doudoroff pathway) |
| E. coli | pgi: glucose-6-phosphate | OE | Increase carbon flux |
| | isomerase | | from G6P to F6P |
| E. coli | pta: phosphate | OE | Increase acetyl-CoA |
| | acetyltransferase | | from |
| | | | acetylphosphate |
| E. coli | ldhA: lactate | KO/KD | Reduce lactate |
| | dehydrogenase A | | production from |
| | | | pyruvate |
| E. coli | poxB: pyruvate oxidase | KO/KD | Reduce acetate |
| | | | production from |
| | | | pyruvate |
| E. coli | ackA: acetate kinase | KO/KD | Reduce acetate |
| | | | production from |
| | | | acetylphosphate |
| E. coli | adhE: alcohol | KO/KD | Reduce ethanol |
| | dehydrogenase enzyme | | production from |
| | | | acetyl-CoA |
| E. coli | did: D-lactate | KO/KD | Reduce lactate |
| | dehydrogenase | | production from |
| | | | pyruvate |
| E. coli | aceE: pyruvate | KD | Increase carbon flux |
| | dehydrogenase | | to MEP pathway |
| | | | from pyruvate |
| E. coli | ispA: geranyl | KD | Increase carbon flux |
| | diphosphate | | to DMAPP from IPP |
| | | | and reduce it to GPP |
| | | | from IPP |

With continued reference to FIGS. 3A-B, in one or more embodiments, promoter engineering may be used to improve the translational level of a gene. Promoter engineering modifies the promoter region to increase gene expression levels, thereby improving enzyme production and activity. In one or more embodiments, codon optimization may be used to improve the translational efficiency of a gene. For the purposes of this disclosure, “codon optimization” is a technique used in genetic engineering to improve the expression of a gene in a particular host organism. Codon optimization involves altering the DNA sequence of a gene to use codons that are more frequently preferred by the host organism's translational machinery. A codon optimization process may take into account a codon bias of the host, ensuring that a synthetic gene sequence is translated more efficiently into the desired protein. Codon optimization may enhance the yield and function of a protein, which may be crucial for various applications in biotechnology and synthetic biology. It is worth noting that promoter engineering and codon optimization are distinct yet complementary techniques in genetic engineering. Promoter engineering involves modifying the promoter region of a gene to enhance its expression by improving the binding efficiency of transcriptional machinery. Codon optimization, on the other hand, focuses on altering the coding sequence of a gene to use preferred codons of the host organism, thereby improving translation efficiency. While both aim to increase protein production, promoter engineering targets transcriptional levels, and codon optimization targets translational efficiency. Combining both techniques may in some cases synergistically enhance an overall gene expression.

With continued reference to FIGS. 3A-B, in some cases, a gene encoding an enzyme may be silenced using Hfq-mediated RNA silencing. For the purposes of this disclosure, “Hfq-mediated RNA silencing” is a process wherein the Hfq protein facilitates a regulation of gene expression through RNA interactions, by binding to small regulatory RNAs (sRNAs) and messenger RNAs (mRNAs) to promote a formation of RNA duplexes. This interaction may enhance or inhibit a translation of target mRNAs, leading to gene silencing. In some cases, this process may be useful for post-transcriptional regulation and may be harnessed for genetic engineering, therapeutic interventions, and synthetic biology applications. As nonlimiting examples, to facilitate accumulation of isoprene, Hfq-mediated RNA silencing may be used against geranyl diphosphate synthase (IspA), farnesyl pyrophosphate synthetase (Erg 20), geranyl diphosphate synthase (GPPS), geranylgeranyl diphosphate synthase (BTS1), farnesyl diphosphate synthase (FPPS), malate synthase (encoded by AceB or MLS1), ATP-citrate lyase including AclY, AclB, and AclA, and/or pyruvate dehydrogenase (encoded by PDH) including pyruvate dehydrogenase E1 component (encoded by AceE), among others, to downregulate its activity, consistent with details described above.

With continued reference to FIGS. 3A-B, in some cases, one or more pathways within schemes of embodiments 300a or 300b may be condensed, inhibited, rearranged, or otherwise modified. As a nonlimiting example, one or more genes encoding one or more enzymes may be modified to provide a phosphoketolase workaround, such as by upregulating glycolysis, the pentose phosphate pathway, or the Entner-Doudoroff pathway. As another nonlimiting example, one or more enzymes may be co-localized to create multiple reaction centers in tandem, thereby bypassing one or more competing side reactions, consistent with details described above and disclosed in U.S. Pat. App. Ser. No. 63/713,890 (attorney docket number 1656-002USP1), filed on Oct. 30, 2024, entitled “GENETICALLY ENGINEERED PEPTIDE SUPERSTRUCTURE AND MICROBE FOR PRODUCTION OF ISOPRENE AND METHOD OF PRODUCTION THEREOF”, the entirety of which is incorporated herein by reference.

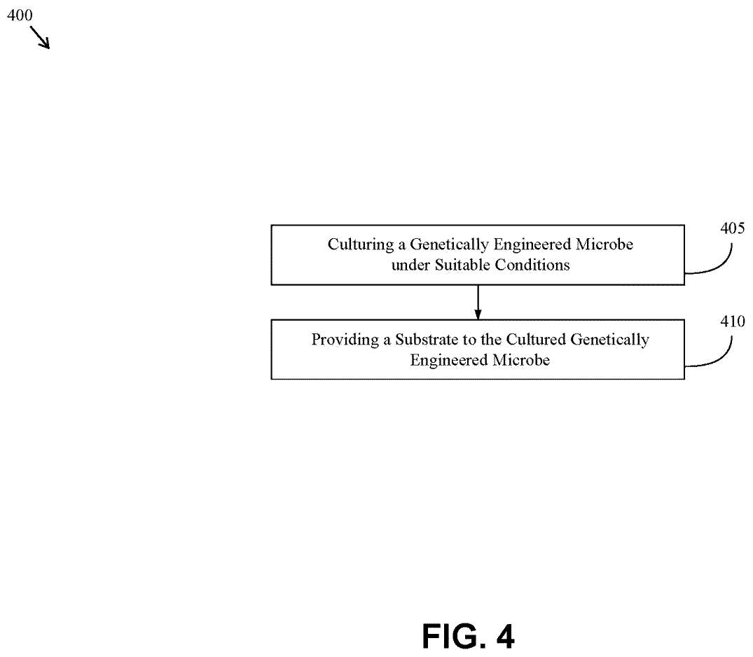

Referring now to FIG. 4, a method 400 of producing a terpene from glycerol is illustrated. At step 405, method 400 includes culturing a genetically engineered microbe under suitable conditions. The genetically engineered microbe includes at least a native nucleic acid sequence encoding at least a native enzyme. The at least a native enzyme is capable of catalyzing one or more steps of a conversion from at least a carbon source to acetyl coenzyme A (A-CoA). The genetically engineered microbe further includes at least a first heterologous nucleic acid sequence encoding at least a first enzyme of a mevalonate (MVA) pathway. The genetically engineered microbe includes at least a second heterologous nucleic acid sequence encoding at least a second enzyme. The at least a second enzyme is capable of catalyzing at least a terpene-producing chemical reaction. This step may be implemented in manners consistent with details described above without limitation. For the purposes of this disclosure, a “suitable condition” is an environmental condition or factor suitable for the growth and/or replication of a microbe. In some cases, a suitable condition may vary from one type of microbe to another. A suitable condition may include without limitation a suitable temperature/temperature range, a suitable pressure/pressure range, a suitable pH or pH range, a suitable ionic strength/range of ionic strength, a suitable osmotic pressure/range of osmotic pressure, a suitable concentration/concentration range of one or more nutrients, a suitable level of metabolic waste/metabolites, among others. A person of ordinary skill in the art, upon reviewing the entirety of this disclosure, will be able to identify suitable conditions specific to one or more microbes described herein.

With continued reference to FIG. 4, at step 410, method 400 further includes providing a substrate to the cultured genetically engineered microbe. This step may be implemented in manners consistent with details described above without limitation.

### EXAMPLES

Optimization of Isoprene Production in Vibrio natriegens Using Mevalonate (MVA) Pathway Engineering

### Experimental Overview

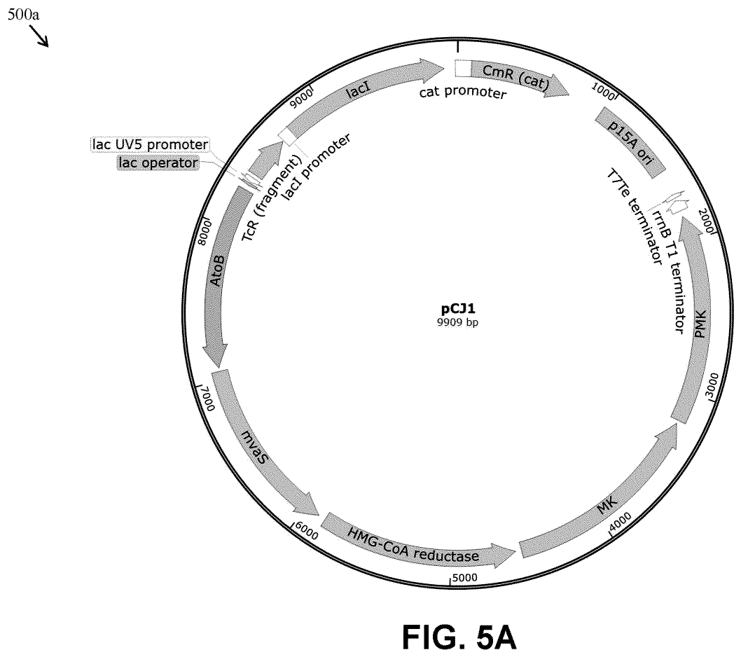

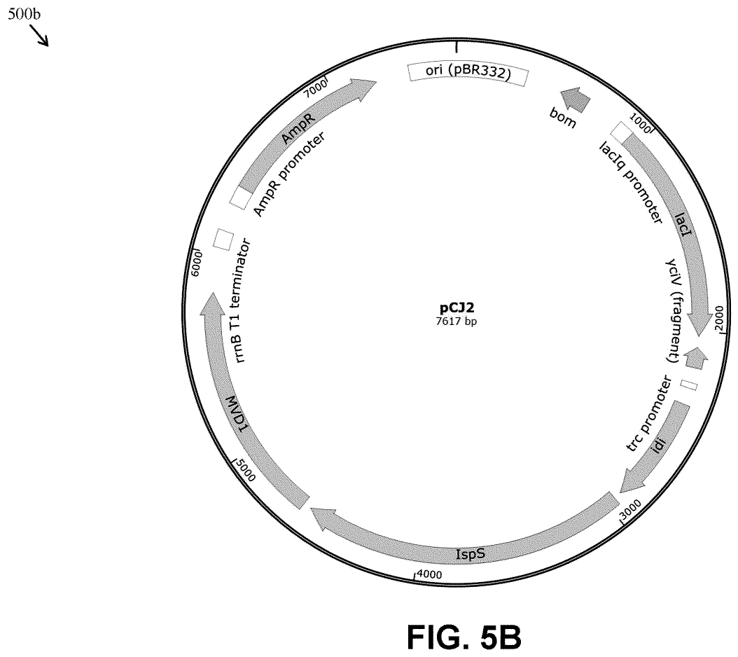

This exemplary study aims to identify suitable conditions for enhancing isoprene production in a genetically modified strain of V. natriegens. Specifically, this study is focused on elucidating the role of carbon sources, growth media composition, and IPTG induction levels. This study used two plasmids, i.e., pCJ1 (see FIG. 5A, embodiment 500a) and pCJ2 (see FIG. 5B, embodiment 500b) to transfect wild-type V. natriegens. These two plasmids contain genes along the mevalonate (MVA) pathway, including atoB (SEQ ID NO: 1), mvaS (SEQ ID NO: 2), mvaA (SEQ ID NO: 3), mk (SEQ ID NO: 4), pmk (SEQ ID NO: 5), idi (SEQ ID NO: 6), ispS (SEQ ID NO: 7), and pmd (SEQ ID NO: 8) genes. Specifically, the pCJ1 plasmid vector includes the atoB (SEQ ID NO: 1), mvaS (SEQ ID NO: 2), mvaA (SEQ ID NO: 3), mk (SEQ ID NO: 4), and pmk (SEQ ID NO: 5) genes, whereas the pCJ2 plasmid vector contains the idi (SEQ ID NO: 6), ispS (SEQ ID NO: 7), and pmd (SEQ ID NO: 8) genes. The two plasmid vectors, which have been optimized for Escherichia coli expression, are 9912 bp and 7619 bp in size, respectively.

### Plasmid Optimization and Transformation

V. natriegens is a promising host for large-scale fermentation applications due to its rapid growth and high metabolic efficiency. The pCJ1/pCJ2 vectors described above were co-transformed into competent cells of V. natriegens via electroporation. After transformation, cells were incubated at 37° C. for 90 to 120 minutes, spread on nutrient agar plates containing chloramphenicol and carbenicillin for selection, and incubated overnight at 37° C. An LBv2 medium was prepared by supplementing an LB medium with 12 g/L sodium chloride, 0.38 g/L potassium chloride, and 2.2 g/L magnesium chloride to support V. natriegens growth under experimental conditions.

### IPTG Induction and Gas Analysis

Colonies from the agar plates were inoculated into an LBv2 medium containing 1 g/L of glucose and incubated at 37° C. in a shaking incubator. After 2 hours, IPTG was added to a series of five samples to reach a concentration of 0.01, 0.05, 0.1, 0.3, and 0.5 mM, respectively. 640 μL of each sample was transferred into Hungate tubes and incubated overnight at 37° C. in a shake incubator. After 20 hours, 200 μL of gas was sampled from the headspace of each tube and analyzed using gas chromatography (GC). Overall isoprene expression titers (measured in peak area units, pA) were similar for all IPTG concentrations, as shown below in Table 2.

TABLE 2
Isoprene Expression Titers for Two V. Natriegens
Samples Transfected by pCJ1/pCJ2 Vectors.

Isoprene (pA)

| IPTG (mM) | pCJ1/pCJ2-2 | pCJ1/pCJ2-3 |
|---|---|---|
| 0.01 | 194 | 190 |
| 0.05 | 255 | 204 |
| 0.1 | 184 | 244 |
| 0.3 | 211 | 196 |
| 0.5 | 229 | 189 |

Follow-Up Experiment with Varying IPTG Concentrations

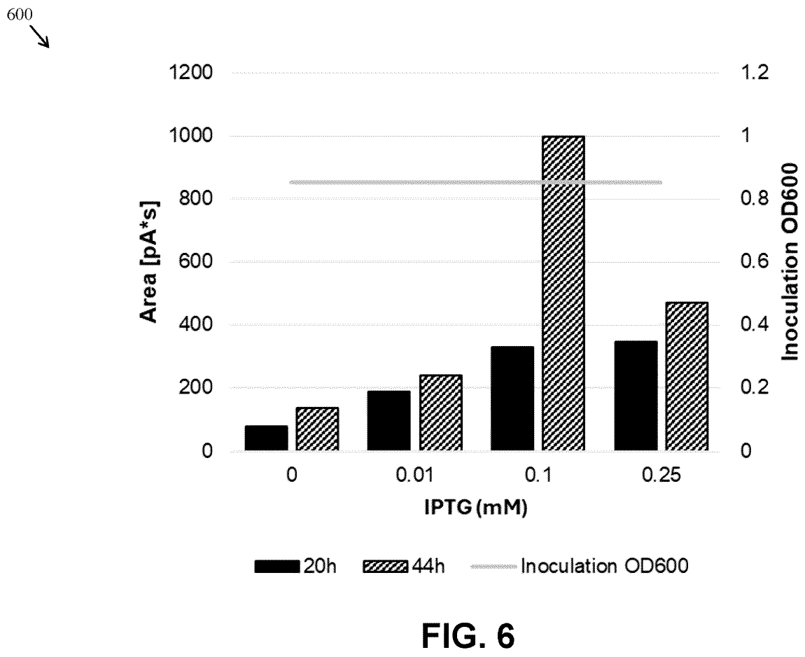

A follow-up experiment explored three different culture parameters: induction OD600 (i.e., the optical density measured at 600 nanometers), culture temperatures, and carbon source. The IPTG induction was conducted when OD600 was 0.85 after 2 hours of inoculation. The cell culture was incubated at 30° C. instead of 37° C., with no additional carbon sources added. The pCJ1/pCJ2-harboring V. natriegens strain was seed-grown overnight in a LBv2 medium and then aliquoted into 5 mL of fresh LBv2 medium the next day. These samples were subsequently incubated at 30° C., in a shake incubator, with each tube containing 0 mM, 0.01 mM, 0.1 mM, and 0.25 mM of IPTG, respectively. Gas samples were collected from the headspace of each sample for GC analysis at 20 and 44 hours, respectively, post-induction. Results from GC showed that 0.1 mM IPTG at 44 hours post-induction led to the highest isoprene production. These data suggest that, while lower concentrations of IPTG may not fully activate gene expression, higher concentrations of IPTG above a certain threshold may lead to a metabolic burden that is detrimental to the production of isoprene. Experimental parameters and corresponding isoprene expression titers are summarized in Table 3 and FIG. 6, results 600.

TABLE 3
Exemplary Experimental Parameters and Corresponding
Isoprene Expression Titers.

| | pCJ1/pCJ2 | | |
|---|---|---|---|
| | Isoprene (pA) | | Inoculation |
| IPTG (mM) | 20 h | 44 h | OD600 |
| 0 | 77 | 135 | 0.85 |
| 0.01 | 186 | 238 | 0.85 |
| 0.1 | 328 | 996 | 0.85 |
| 0.25 | 347 | 469 | 0.85 |

### Testing Modified Media and Carbon Sources

To optimize isoprene production, media compositions were further modified, and different carbon sources were tested. An LB3 medium was prepared by adding 20 g/L sodium chloride to an LB medium. Glucose, glycerol, and maltose were selected as carbon sources for comparison, each at working concentrations of approximately 1 g/L, 5 g/L, and 2 g/L, respectively.

Glucose is a six-carbon monosaccharide and generally a readily accessible carbon source. 1 g/L of glucose provides approximately 0.033 mol/L of carbon. However, in this exemplary study, glucose was less effective as a carbon source when used for isoprene production, possibly due to metabolic interference within V. natriegens that potentially inhibits efficient MVA pathway activation. Glycerol, a three-carbon molecule, was added at 0.5 v/v % 5 g/L as a carbon source, with a concentration calculated to be 6.3 g/L (equivalent to 0.2 mol/L of carbon). The high carbon content and energy density may enable glycerol to efficiently contribute to acetyl-CoA generation, which is an essential precursor in isoprene synthesis, consistent with details described above in this disclosure. Finally, maltose, a disaccharide containing two glucose units, was tested as a carbon source. A solution containing 2 g/L maltose provided approximately 0.07 mol/L of carbon. While maltose may potentially serve as a steady energy source, the additional metabolic steps required for maltose breakdown may have limited its efficiency in producing isoprene.

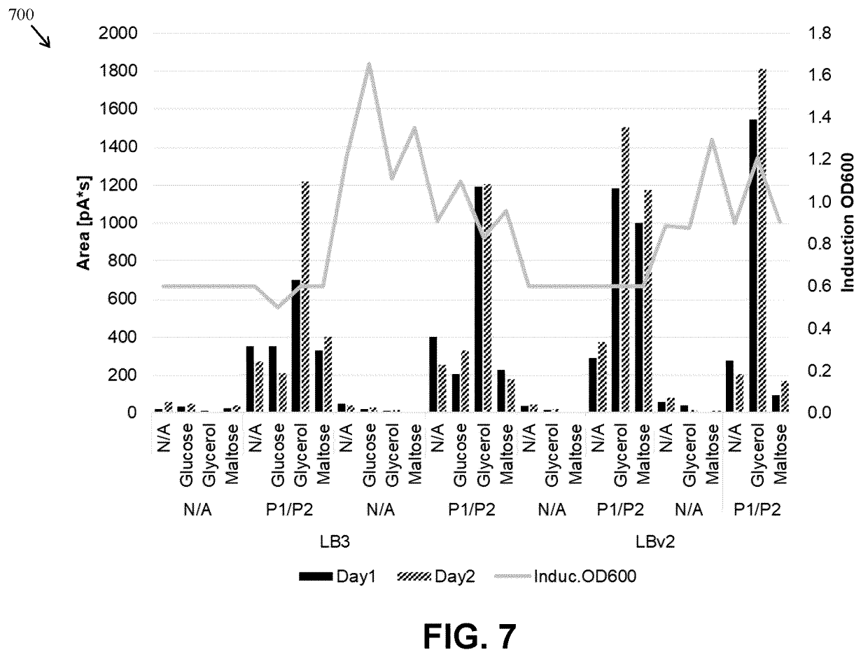

Gas chromatography (GC) analysis on Days 1 and 2 confirmed that samples with glycerol consistently produced higher isoprene titers than those treated with glucose or maltose, likely due to the more efficient metabolic pathways of V. natriegens when utilizing glycerol as a carbon source. This result represents glycerol as a more suitable carbon source for isoprene production in V. natriegens under these experimental conditions. Experimental parameters and corresponding isoprene expression titers are summarized in Table 4 and FIG. 7, results 700.

TABLE 4
Exemplary Experimental Parameters and Corresponding
Isoprene Expression Titers.

| | | | Concen- | | | |
|---|---|---|---|---|---|---|
| | | | tration | Induction | Isoprene (pA) | |
| Media | Vectors | Carbon | (g/L) | OD600 | Day 1 | Day 2 |
| LB3 | N/A | N/A | 0 | 0.6 | 21 | 59 |
| | | Glucose | 1 | 0.6 | 33 | 52 |
| | | Glycerol | 5 | 0.6 | 10 | 7.5 |
| | | Maltose | 2 | 0.6 | 25 | 41 |
| | pCJ1/ | N/A | 0 | 0.6 | 351 | 272 |
| | pCJ2 | Glucose | 1 | 0.5 | 352 | 209 |
| | | Glycerol | 5 | 0.6 | 699 | 1215 |
| | | Maltose | 2 | 0.6 | 330 | 399 |
| | N/A | N/A | 0 | 1.2 | 51 | 40 |
| | | Glucose | 1 | 1.7 | 20 | 28 |
| | | Glycerol | 5 | 1.1 | 10 | 13 |
| | | Maltose | 2 | 1.4 | 2 | 5 |
| | pCJ1/ | N/A | 0 | 0.9 | 400 | 255 |
| | pCJ2 | Glucose | 1 | 1.1 | 206 | 329 |
| | | Glycerol | 5 | 0.8 | 1190 | 1205 |
| | | Maltose | 2 | 1.0 | 227 | 176 |
| LBv2 | N/A | N/A | 0 | 0.6 | 36 | 46 |
| | | Glycerol | 5 | 0.6 | 14 | 18 |
| | | Maltose | 2 | 0.6 | 5 | 4 |
| | pCJ1/ | N/A | 0 | 0.6 | 289 | 373 |
| | pCJ2 | Glycerol | 5 | 0.6 | 1181 | 1506 |
| | | Maltose | 2 | 0.6 | 1002 | 1171 |
| | N/A | N/A | 0 | 0.9 | 60 | 82 |
| | | Glycerol | 5 | 0.9 | 41 | 13 |
| | | Maltose | 2 | 1.3 | 4 | 9 |
| | pCJ1/ | N/A | 0 | 0.9 | 275 | 204 |
| | pCJ2 | Glycerol | 5 | 1.2 | 1542 | 1809 |
| | | Maltose | 2 | 0.9 | 95 | 168 |

### CONCLUSION

Glycerol at a concentration of 0.5-1 v/v % was identified as an efficient carbon source for isoprene production in V. natriegens, with superior performance compared to glucose and maltose. Glucose appeared to inhibit isoprene production, likely due to an impact on metabolic flux, and additional metabolic requirements posed by maltose potentially limited its effectiveness at higher concentrations. Glycerol's high energy yield and favorable influence on isoprene levels make it a suitable carbon source under these conditions.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A genetically engineered microbe capable of producing a terpene, the genetically engineered microbe comprising:
at least a native nucleic acid sequence encoding at least a native enzyme capable of catalyzing one or more steps of a conversion of a carbon source to acetyl coenzyme A (A-CoA), wherein the carbon source comprises glycerol, wherein the conversion includes one or more steps utilizing glycerol as a substrate, wherein the genetically engineered microbe comprises a genetically engineered bacterium, and wherein the genetically engineered bacterium comprises at least Vibrio natriegens;
at least a first heterologous nucleic acid sequence encoding at least a first enzyme of a mevalonate (MVA) pathway; and
at least a second heterologous nucleic acid sequence encoding at least a second enzyme capable of catalyzing at least a terpene-producing chemical reaction.

2. (canceled)

3. (canceled)

4. The genetically engineered microbe of claim 1, wherein glycerol has a concentration of at least 0.1 g/L and no greater than 10 g/L.

5. The genetically engineered microbe of claim 1, wherein the at least a carbon source further comprises glucose having a concentration of at least 0.1 g/L and no greater than 10 g/L.

6. The genetically engineered microbe of claim 1, wherein the at least a carbon source further comprises maltose having a concentration of at least 0.1 g/L and no greater than 10 g/L.

7. The genetically engineered microbe of claim 1, wherein the at least a first heterologous nucleic acid sequence includes one or more genes selected from a group consisting of atoB gene, mvaS gene, mvaA gene, mk gene, pmk gene, idi gene, and pmd gene.

8. The genetically engineered microbe of claim 1, wherein the at least a first heterologous nucleic acid sequence includes one or more genes selected from a group consisting of ERG10 gene, ERG13 gene, HMG1 gene, HMG2 gene, ERG12 gene, ERG8 gene, MVD1 gene, and IDI1 gene.

9. The genetically engineered microbe of claim 1, wherein the at least a first enzyme includes at least a functional fragment of one or more enzymes selected from a group consisting of 3-hydroxy-3-methylglutaryl-coenzyme A reductase, acetyl-CoA acetyltransferase, 3-hydroxy-3-methylglutaryl-CoA synthase, HMG-CoA reductase, mevalonate kinase, phosphomevalonate kinase, diphosphomevalonate decarboxylase, and isopentenyl diphosphate isomerase.

10. The genetically engineered microbe of claim 1, wherein the at least a second heterologous nucleic acid sequence includes an ispS gene encoding at least a functional fragment of isoprene synthase (IspS).

11. The genetically engineered microbe of claim 1, wherein the terpene-producing chemical reaction utilizes dimethylallyl pyrophosphate (DMAPP) as a reactant.

12. The genetically engineered microbe of claim 1, wherein at least a metabolic pathway of the genetically engineered microbe is inducible by isopropyl β-D-thiogalactopyranoside (IPTG) having a concentration of at least 0.05 millimolar and no greater than 0.3 millimolar.

13. The genetically engineered microbe of claim 1, wherein at least a metabolic pathway of the genetically engineered microbe is inducible by isopropyl β-D-thiogalactopyranoside (IPTG) having a concentration of at least 0.01 millimolar and no greater than 0.5 millimolar.

14. A method of producing a terpene, the method comprising:
culturing a genetically engineered microbe under suitable conditions, the genetically engineered microbe comprising:
at least a native nucleic acid sequence encoding at least a native enzyme capable of catalyzing one or more steps of a conversion of a carbon source to acetyl coenzyme A (A-CoA), wherein the carbon source comprises glycerol, wherein the genetically engineered microbe comprises a genetically engineered bacterium, and wherein the genetically engineered bacterium comprises at least Vibrio natriegens;
at least a first heterologous nucleic acid sequence encoding at least a first enzyme of a mevalonate (MVA) pathway; and
at least a second heterologous nucleic acid sequence encoding at least a second enzyme capable of catalyzing at least a terpene-producing chemical reaction; and
providing a substrate to the cultured genetically engineered microbe, wherein the conversion includes one or more steps utilizing glycerol as the substrate.

15. (canceled)

16. (canceled)

17. The method of claim 14, wherein glycerol has a concentration of at least 0.1 g/L and no greater than 10 g/L.

18. The method of claim 14, wherein the at least a carbon source further comprises glucose having a concentration of at least 0.1 g/L and no greater than 10 g/L.

19. The method of claim 14, wherein the at least a carbon source further comprises maltose having a concentration of at least 0.1 g/L and no greater than 10 g/L.

20. The method of claim 14, wherein the at least a first heterologous nucleic acid sequence includes one or more genes selected from a group consisting of atoB gene, mvaS gene, mvaA gene, mk gene, pmk gene, idi gene, and pmd gene.

21. The method of claim 14, wherein the at least a first heterologous nucleic acid sequence includes one or more genes selected from a group consisting of ERG10 gene, ERG13 gene, HMG1 gene, HMG2 gene, ERG12 gene, ERG8 gene, MVD1 gene, and IDI1 gene.

22. The method of claim 14, wherein the at least a first enzyme includes at least a functional fragment of one or more enzymes selected from a group consisting of 3-hydroxy-3-methylglutaryl-coenzyme A reductase, acetyl-CoA acetyltransferase, 3-hydroxy-3-methylglutaryl-CoA synthase, HMG-CoA reductase, mevalonate kinase, phosphomevalonate kinase, diphosphomevalonate decarboxylase, and isopentenyl diphosphate isomerase.

23. The method of claim 14, wherein the at least a second heterologous nucleic acid sequence includes an ispS gene encoding at least a functional fragment of isoprene synthase (IspS).

24. The method of claim 14, wherein the terpene-producing chemical reaction utilizes dimethylallyl pyrophosphate (DMAPP) as a reactant.

25. The method of claim 14, wherein at least a metabolic pathway of the genetically engineered microbe is induced by isopropyl β-D-thiogalactopyranoside (IPTG) having a concentration of at least 0.05 millimolar and no greater than 0.3 millimolar.

26. The method of claim 14, wherein at least a metabolic pathway of the genetically engineered microbe is induced by isopropyl β-D-thiogalactopyranoside (IPTG) having a concentration of at least 0.01 millimolar and no greater than 0.5 millimolar.
